(19) United States
(12) Patent Application Publication
(10) Pub. No.: US 2008/0063598 A1
(43) Pub. Date: Mar. 13, 2008

(54) Humanized Antibody

(75) Inventors: Paul Averback, Beaconsfield (CA); Jack Gemmell, Mississauga (CA)

Correspondence Address: GOODWIN PROCTER LLP, 901 NEW YORK AVENUE, N.W., WASHINGTON, DC, 20001, US

(73) Assignee: Nymox Pharmaceutical Corporation

(21) Appl. No.: 11/841,358

(22) Filed: Aug. 20, 2007

Related U.S. Application Data
(63) Continuation of application No. 11/050,435, filed on Feb. 4, 2005, now abandoned.
(60) Provisional application No. 60/541,944, filed on Feb. 6, 2004.

(51) Int. Cl. C07K 16/30; A61K 39/395; A61K 51/10; C07K 7/08; C12N 15/11; C12N 15/63; C12N 5/16; C12N 15/13; C12N 1/00; C07K 7/06; A61P 35/00

(52) U.S. Cl. 424/1.49; 424/133.1; 424/178.1; 424/9.1; 435/243; 435/320.1; 435/328; 435/7.23; 530/326; 530/327; 530/328; 530/329; 530/387.3; 536/23.1; 536/23.53

(57) ABSTRACT

Novel humanized and chimeric antibodies, humanized antibody fragments, polypeptides sequences of such antibodies and derivatives thereof that specifically bind AF-20 are provided as well as methods for their manufacture. These humanized and chimeric antibodies, antibody fragments and polypeptide sequences are useful in the treatment of cancers that express AF-20, as well as for diagnostic purposes, e.g., for in vivo imaging of tumors or cancer cells that express AF-20.

This application is a continuation of U.S. application Ser. No. 11/050,435 entitled: “Humanized Antibody,” filed on Feb. 4, 2005 which claims priority under 35 U.S.C. §119 to Provisional Patent Application No. 60/541,944 entitled: “Humanized Antibody,” filed on Feb. 6, 2004, the disclosures of which are incorporated by reference herein in their entireties.

### FIELD OF THE INVENTION

Embodiments of the present invention relate to humanized and chimeric antibodies, fragments, polypeptides or derivatives thereof that are capable of binding to adenocarcinoma cell antigen AF-20, which is associated with carcinoma cells, and especially with hepatocarcinoma cells, and adenocarcinoma cells of the colon and lung.

### DESCRIPTION OF RELATED ART

Cancer is the second leading cause of death in the United States. Despite progress to date, the incidence of cancer per 100,000 in the U.S. population has not declined since 1950; in fact it has slightly increased. Accordingly, there remains a pressing need for effective cancer treatments and a concomitant need for new approaches to diagnosis, assessment and monitoring of cancer cases.

Of particular need are methods to target metastases for prevention and/or treatment. One of the most devastating aspects of cancer is the propensity of cells from malignant neoplasms to disseminate from their primary site and metastasize at distant organs. Despite advances in surgical treatment of primary neoplasms and aggressive therapies, most cancer patients die as a result of metastatic disease. Animal tests indicate that about 0.01% of circulating cancer cells from solid tumors establish successful metastatic colonies (Fidler, 1993). Monoclonal antibodies (hereinafter “moAbs”) specific to tumor-associated antigens offer great promise in the treatment of cancer and in the targeting of metastates.

Hepatocellular Caracinoma—Hepatocellular carcinoma (hereinafter “HCC”) is one of the leading causes of cancer-related death in the world. HCC accounts for more than 80 percent of liver carcinomas. There is a wide variation in the incidence of HCC in different parts of the world, with the incidence in Asia and Africa being at least ten times that of the United States. A number of factors have been identified as being of potential importance in the etiology of this disease. Chronic liver diseases such as chronic hepatitis B virus (HBV) or hepatitis C virus (HCV) infection or cirrhosis predispose individuals toward HCC. High HBV and HCV infection rates in many parts of the world, especially in Asia and Africa, may account for HCC being one of the most common human malignancies. Aflatoxins, present in certain foodstuffs such as tree nuts and peanuts, are also thought to predispose an individual toward HCC. Since liver cancer occurs 2-4 times more frequently in men than in women, hormonal factors may be important in the etiology of HCC.

The prognosis for HCC is poor, with death often resulting within 3-6 months. Only about six percent of those diagnosed with HCC will survive five years. Localized HCC can be treated by surgery, through a partial hepatectomy or total hepatectomy with liver transplation or, if unresectable, through ablative therapies such as radiofrequency ablation, cryosurgery or percutaneous ethanol injection or chemotherapies administered by means of hepatic arterial infusion or chemoembolization. Only ten to twenty percent of all HCC surgeries succeed in removing cancerous tissue entirely. Advanced HCC may be treated with systemic chemotherapy or radiation therapy but with limited effect and little success.

The symptoms of HCC typically become apparent only late in the disease, making treatment more difficult. Diagnostic tests include an alpha-fetoprotein (AFP) blood test. A high AFP test only indicates the possibility of liver cancer; it cannot confirm the diagnosis. Between 50 and 75 percent of people suffering from primary liver cancer have high levels of AFP. Other conditions, most notably cirrhosis, chronic hepatitis infections, and several other cancers also produce high levels of AFP. In addition to the AFP blood test, a number of other tests that measure enzyme, bilirubin, and protein levels can identify possible liver dysfunction. Diagnostic imaging such as a liver scan, computed tomography (CT) scan, ultrasound, or magnetic resonance imaging (MRI) can identify potential liver tumors and sites for biopsy. None of these tests alone can be used to diagnose hepatic cancer. A liver biopsy still remains the best way to reach a definite diagnosis of HCC. The procedure is generally very safe, although in less than 0.5 percent of cases, a fatal hemorrhage can result, as some tumors are connected to multiple blood vessels.

Lung Cancer—Lung cancer is the leading cause of cancer death in the United States and one of the leading causes of death throughout the world. The overall 5-year survival for affected individuals (about 13 percent) has not changed significantly over the past 25 years. After many years of dramatic increase, the incidence of this disease appears to have leveled off. Cigarette smoking is the cause of about 90% of lung cancer cases in men and about 80% of cases in women. The greater the quantity and duration of smoking, the greater the iisk of developing lung cancer. About 10 to 12% of all smokers eventually develop lung cancer.

Primary lung carcinomas are divided into two major types: non-small cell carcinoma and small cell carcinoma. Non-small cell carcinoma is more common and has a better prognosis than small cell carcinoma. There are three main classes of non-small cell lung carcinoma: squamous cell carcinoma (also called epidermoid carcinoma), adenocarcinoma, and large cell carcinoma. Adenocarcinoma is the most common type of lung cancer, accounting for 30-35% of all cases.

The prognosis for primary lung cancer is poor. Less than one percent of individuals having small-cell carcinoma survive five years after the diagnosis of the disease. In contrast, the prognosis for individuals having non-small-cell carcinoma depends on the staging of the cancer, and particularly on the presence or absence of distant metastases. Distant metastases are associated with a five year survival rate of under five percent. Liver tissue is a common site for such metastases.

Small cell carcinomas are presently treated by combinations of surgical resection, radiation therapy and chemotherapy. Despite such aggressive treatments, the prognosis of the disease is extremely poor. The treatment of choice for non-small-cell carcinoma of the lung involves surgical resection of the cancerous lesion. Unfortunately, such surgical operations are possible only in the earliest stages of the disease, and even with surgery the five year survival rate is on the order of 25% to 40%. Although radiation therapy can be applied to treat non-small-cell carcinomas in latter stages, the prognosis of this therapy is poor. Chemotherapy has limited effectiveness for non-small cell carcinoma but can significantly increase duration of survival in metastatic non-small cell carcinoma.

The diagnosis and detection of lung cancers is facilitated by, inter alia, chest X-rays, CT scans of the lungs, bronchoscopy and biopsy.

Colorectal Carcinomas—Colon and rectal cancers are the second leading cause of cancer-related death, accounting for approximately 20% of all cancer deaths in the United States. The five year survival rate is approximately 63%; distant metastases are associated with a much lower survival rate of less than 10%. Approximately 60 percent of those patients diagnosed with colorectal cancer will develop hepatic metastases for which the therapeutic gold standard remains hepatic resection. Despite surgical treatment, the majority of patients after liver resection will develop recurrences and of these recurrences, approximately fifty percent will be within the liver. Almost all colorectal cancers are adenocarcinomas.

Delay in diagnosis significantly affects the prognosis for colorectal carcinoma. If detected early, colorectal cancer often can be successfully treated. Thus, for example, patients whose tumor is confined to the bowel wall generally have an excellent chance for cure following surgical resection (five-year survival rate >95%). Where the tumor has extended to the serosa and mesenteric fat, however, the five-year survival rate following resection declines to 80%. Lymph node metastases reduce the five-year survival rate to 40%, while distant metastases (e.g., liver, lung, bone, brain) reduce the five-year survival rate to below 10%. Because symptoms of colorectal carcinoma are frequently vague and nonspecific in the early stages of the disease, detection is often delayed. As a result, the cancer often is so well established by the time a positive diagnosis is made that a cure is difficult or impossible. Colorectal carcinomas generally respond poorly to chemotherapy. Although palliation may be effected, chemotherapy has not been shown to prolong the lives of patients diagnosed as having colorectal cancer, especially when the disease is widely disseminated.

The U.S. Preventive Services Task Force (USPSTF) strongly recommends that clinicians screen men and women 50 years of age or older for colorectal cancer. The USPSTF found good evidence that periodic fecal occult blood testing (FOBT) reduces mortality from colorectal cancer and fair evidence that sigmoidoscopy alone or in combination with FOBT reduces mortality. However, commonly used screening tests for colorectal carcinoma can generate false positives and may contribute to delayed detection of the disease through false negatives. For example, FOBT detects occult blood in the stool which requires that a colonic malignancy be advanced to the bleeding stage before it can be detected. Sigmoidoscopy requires that any colorectal carcinoma be visible, and diagnosis may be complicated by the presence of other lesions such as hemorrhoids, polyps, and proctitis. Colonoscopy has similar drawbacks.

Monoclonal antibodies (“moAbs”) specific to tumor-associated antigens offer great promise in the research, diagnosis, monitoring and treatment of cancers. However, significant practical problems have stood in the way of their widespread in vivo use in humans and other mammals.

A major concern is that monoclonal antibodies of non-human origin often are immunogenic, thereby limiting their effectiveness and, in some cases, causing dangerous allergic reactions. Most moAbs are of murine origin, and have generally been found to be immunogenic when injected into humans. The immune response to such foreign moAbs includes the production of specific, high affinity antibodies which bind to and effect elimination of the moAbs, thereby substantially reducing the moAb's effectiveness by promoting its clearance from the body and inhibiting its ability to bind to the targeted tumor-associated antigen.

Many methods that may reduce the immunogenicity of non-human antibodies are known. These include:
the creation of chimeric antibodies by attaching the variable regions in the heavy and light chains of the non-human antibody onto the constant regions of a human antibody as described by Cabilly, et al. in U.S. Pat. No. 4,816,567; Morrison, S. L. et al., Proc. Natl. Acad. Sci. USA 81:6851-6855 (1984); Boulianne, G. L. et al., Nature 312;643-646 (1984); Neuberger, M. S. et al., Nature 314:268-270 (1985).
the creation of humanized antibodies by the substitution of non-human complementarity determining regions (CDRs) or CDR sequences with corresponding segments of a human antibody (also known as CDR grafting) as described by Winter in U.S. Pat. No. 5,225,539, and in Jones, P. T. et al., Nature 321:522-525 (1986); Riechmann, L. et al, Nature 332:323-327 (1988); Verhoeyen, M. et al., Science 239:1534-1536 (1988). This may also entail the substitution of some FR residues in the human antibody with residues from analogous sites in non-human antibody in order to retain antigen binding as described, for example, by Queen, et al. in U.S. Pat. Nos. 5,530,101, 5,585,089, 5,693,762, 6,180,370, Carter, et al. in U.S. Pat. Nos. 6,054,297, 6,407,213 and 6,639,055, Adair in U.S. Pat. No. 6,632,927 and Winter in U.S. Pat. No. 6,548,640.
the creation of humanized antibodies by selective substitution of residues in the variable regions of the non-human antibody (also known as veneering or resurfacing) as described, for example, by Pedersen, et al. in U.S. Pat. No. 5,639,641, Studnicka, et al. in U.S. Pat. Nos. 5,766,886 and 5,821,123, and Carr et al, in U.S. patent application Ser. No. 10/300215, now U.S. Pat. No. 7,125,689.
the linking of an antibody or antibody fragment to auto-antigenic sequences which render the non-human antibody or antibody fragment less immunogenic as described by Jordan, et al. in U.S. Pat. No. 6,652,863.

The number of methods of creating chimeric or humanized antibodies is indicative of the difficulty encountered in developing appropriate antibody candidates. It is not uncommon to find that the resulting antibody has too low an affinity or specificity to the targeted tumor-associated antigen, still elicits an unfavorable immune response, is too difficult to express in practicable amounts or has other unfavorable characteristics.

Another major concern relates to the tumor-associated antigen that the antibody targets. The efficacy of the treatment depends on the specificity of the antigen to the tumor, its role in tumor growth and its expression by tumor cells. The tumor-associated antigen may be widely expressed in normal tissues, thereby requiring higher effective doses of the treatment and increasing the risk of unwanted side-effects. The antigen may be expressed by only a small percentage of tumor, by only a percentage of the cells in any one tumor, or both. The antigen may only be secreted by the tumor cells and not expressed on the tumor cell surface, malting targeting of cytotoxic therapies more difficult, if not impossible. Binding to an antigen expressed on the cell surface may not result in the internalization of a cytotoxic agent into the cell or in the desired inhibition of function.

Despite these obstacles, and after many attempts, a small number of monoclonal antibodies have achieved regulatory approval. Approved antibody-based therapeutics include:
trastuzumab (Herceptin®), a humanized monoclonal antibody (moAb) that binds human epidermal growth factor receptor 2 (HER2), thereby inhibiting tumor cell proliferation and migration in metastatic breast cancers that overexpress HER2. It currently is indicated for use either alone or in combination with paclitaxel, a chemotherapeutic agent, for the treatment of metastatic breast cancers overexpressing HE2 protein. Herceptin® is in clinical trials for treatment of nonmetastatic breast cancer and also is being studied in clinical trials for other types of cancer that may overexpress the HER-2 protein, including osteosarcoma, non-small cell lung cancer and cancers of the pancreas, salivary gland, colon, prostate, endometrium, and bladder. It also is being investigated for use when conjugated to a cytotoxin such as geldanamycin
alemtuzumab (Campath®), a humanized moAb that binds the CD52 antigen. It is currently indicated for use for the treatment of refractory B-cell chronic lymphocytic leukemia and is being investigated for use for the treatment of other chronic lymphocytic and chronic myelogenous leukemias.
gemtuzumab (Mylotarg®), a humanized moAb that binds CD33, which is a protein expressed in about 90% of cases of acute myeloid leukemia (AML). Mylotarg® is conjugated to a bacterial toxin, calicheamicin which induces DNA strand breaks and cellular apoptosis. It is indicated for use for the treatment of AML.
rituximab (Rituxan®), a chimeric moAb that binds CD20, an antigen found on the surface of mature B cells, thereby marking the cells for destruction by the body's immune system. It is indicated for use for the treatment of relapsed or refractory, low-grade or follicular B-cell non-Hodgkin's lymphoma (NHL). It is also under investigation for use for the treatment of B-cell lymphoma and chronic lymphocytic leukemia.
ibritumomab (Zevalin®), a murine moAb that is conjugated to a beta-emitting radioisotope, Yttrium-90 (⁹⁰Y), also binds CD20 and induces cellular damage in the target and neighboring cells. It is indicated for use in combination with Rituxan® for the treatment of NHL.
tositumomab (Bexxar®), a murine moAb that also binds CD20 and is conjugated to another radioisotope, Iodine-131 (¹³¹I). It is indicated for use for the treatment of relapsing NHL following chemotherapy with Rituxan®.
edrecolomab (Panorex®), a murine moAb that binds epithelial cell adhesion molecule. It is approved for use in Europe for the treatment of colorectal cancer, and is in Phase III clinical trials in the U.S. for colorectal and breast cancer.

Other therapeutic antibodies in phase III trials include:
cetuximab (Erbitux®), a chimeric moAb that binds epidermal growth factor receptor (EGFR) and is being investigated for the treatment of cancers of the head and neck, non-small cell lung cancer, colorectal cancer, breast cancer, and cancers of the pancreas and prostate.
bevacizumab (Avastin®), a humanized moAb that binds vascular endothelial growth factor (VEGF) and is being investigated for the treatment of metastatic colorectal cancer, breast cancer, and non-small cell lung cancer.

Humanized antibodies and antibody fragments also are useful in the generation of optimized new therapeutics through the application of methods designed to improve their characteristics and performance such as phage display, bacterial or yeast cell surface display (e.g. Kieke, et al., U.S. Pat. No. 6,300,065 and Wittrup, et al., U.S. Pat. No. 6,423,538), and other directed molecular evolution technologies (e.g. Co, et al., U.S. Pat. No. 5,714,350).

Chimeric and humanized antibodies and antibody fragments also have use in the diagnosis, staging, and treatment monitoring of cancer. Enhanced in vivo half-lives and reduced immunogenicity make moAbs potentially more practical for in vivo immunoimaging where a detectable label such as a radionuclide or resonance imaging agent is conjugated to the antibody or antibody fragment. Such antibodies and antibody fragments also have use in determining whether the tumor expresses the antigen before commencing treatment, determining sites for local administration of the antibody, and checking for recurrence after treatment. Like other antibodies, such antibodies also have use in immunohistology and immunoassays.

The AF-20 Tumor Associated Antigen is a rapidly internalized 180 kDa homodimeric cell-surface glycoprotein that is abundantly expressed on human hepatocellular carcinoma (HCC) cells, as well as distant metastases such as adenocarcinoma of the lung cells and colorectal carcinoma cells. The AF-20 antigen was found by means of a high affinity murine monoclonal antibody (AF-20 moAb) discovered by immunizing mice with the hepatocellular carcinoma cell line FOCUS and screening hybridomas for antibody activity on a panel of cell lines. AF-20 antigen has not been found to be expressed in normal liver tissue adjacent to HCC tissue, nor in other normal tissues with the exception of the adrenal gland. Low-level expression of AF-20 antigen has been found on a subpopulation of cells in the zona glomenilosa of the adrenal gland and on crypt cells of the small intestinal tract. (See Wands, et al., U.S. Pat. No. 5,703,213; Wilson et al., “Cell-surface changes associated with transformation of human hepatocytes to the malignant phenotype,” Proc Natl Acad Sci USA (1988 85:3140-4); Takahashi et al., “In vivo expression of two novel tumor-associated antigens and their use in immunolocalization of human hepatocellular carcinoma,” Hepatology (1989; 9:625-34); Moradpour et al., “Specific targeting of human hepatocellular carcinoma cells by immunoliposomes in vitro,” Hepatology (1995; 22:1527-37); Wands et al., “Immunological approach to hepatocellular carcinoma,” J Viral Hepat (1997; 4 Suppl 2:60-74); Mohr et al., “Targeted gene transfer to hepatocellular carcinoma cells in vitro using a novel monoclonal antibody-based gene delivery system,” Hepatology (1999; 29:82-9); Yoon et al., “Targeting a recombinant adenovirus vector to HCC cells using a bifunctional Fab-antibody conjugate,” Biochem Biophys Res Commun. (2000; 272:497-504); Palumbo et al., “Human aspartyl (asparaginyl) beta-hydroxylase monoclonal antibodies: potential biomarkers for pancreatic carcinoma,” Pancreas (2002; 25:39-44); Yoon et al., “Targeted cancer therapy using chimeric immunotoxin of AF-20 monoclonal antibody with Pseudomonas exotoxin for hepatocellular carcinoma,” (unpublished abstract, June 2002); the disclosures of each of which are herein incorporated by reference).

The AF-20 moAb has shown potential both as an immunotargeting agent and as an immunoimaging agent. AF-20 moAb radiolabeled with ¹²⁵I has been successfully used in nude mice models for in vivo radioimaging to localize a hepatitis B virus-related hepatocellular carcinoma cell line (FOCUS) grown as subcutaneous tumors. Nuclear imaging studies showed sharp visualization of tumor tissue, demonstrating good specificity and sensitivity of AF-20 moAb as a potential immunotargeting or immunoimaging agent.

AF-20 moAb has been found to be rapidly internalized by HCC cells, making it a good candidate for the targeted delivery of a cytotoxic agent or gene therapy to tumor cells that express the AF-20 antigen. Antibodies to tumor-associated antigens which are not able to internalize within the tumor cells to which they bind are generally not useful for such targeted delivery, since they are not able to reach their site of action within the cell. In one study (Yoon 2002 supra), AF-20 moAb conjugated to Pseudomonas exotoxin was found to have potent anti-tumor activity in vitro with HCC cells and in vivo with nude mice with HCC xenografts. In another approach (Moradpour supra), AF-20 moAb was covalently coupled to liposomes containing carboxyfluorescein. AF-20-immunoliposomes specifically bound to HCC and other human cancer cell lines expressing the AF-20 antigen and were rapidly internalized at 37° C. Interaction of AF-20-conjugated liposomes with these cell lines was between 5 and 200 times greater than that of unconjugated liposomes, whereas no difference was observed between control liposomes bearing a nonrelevant antibody and unconjugated liposomes. Kinetic analysis showed rapid association of AF-20 immunoliposomes with target cells, with saturation conditions being reached after 60 minutes.

AF-20 moAb also has been used to develop experimental targeted gene delivery systems. In one such approach, a specific adenoviral gene delivery system consisting of a bifunctional Fab-antibody conjugate (2Hx-2-AF-20) was generated through AF-20 moAb crosslinkage to an anti-hexon antibody Fab fragment. The conjugate complex was found to be rapidly internalized at 37° C., and enhanced levels of reporter gene expression was observed in AF-20 antigen positive HCC cells, but not in AF-20 antigen negative control cells. In another approach, AF-20 moAb was coupled to a DNA-binding cationic amphiphile, cholesteryl-spermine, for gene delivery to hepatocellular carcinoma (HCC) cells. Binding and internalization of AF-20-cholesteryl-spermine was confirmed by fluorescence microscopy using fluorescein isothiocyanate (FITC)-labeled anti-mouse IgG antibody. Transfection of luciferase or beta-galactosidase reporter genes complexed to AF-20-cholesteryl-spermine resulted in high levels of gene expression in AF-20 antigen-positive tumor cells.

As the above discussion shows, there is a pressing need for cancer treatments, particularly for hepatocellular carcinoma, adenocarcinoma of the lung and colorectal carcinoma. The murine monoclonal antibody, AF-20 moAb, offers promise as a means of delivering targeted therapy to these tumors and their metastases, but in order to be effective must have reduced immunogenicity, or preferably no immunogenicity. Accordingly there is a need for chimeric and humanized antibodies derived from AF-20 moAb that retain their affinity and specificity for the AF-20 antigen.

The description herein of disadvantages and deleterious properties associated with known compositions, methods, and systems is in no way intended to limit the scope of the invention to their exclusion. Indeed, embodiments of the invention may include portions of, or one or more known compositions, methods, and systems without suffering from the disadvantages and deleterious properties.

### SUMMARY OF THE EMBODIMENTS

Embodiments of this invention are made available by the surprising discovery and development of chimeric and humanized antibodies that retain favorable affinity with AF-20 and offer an important new approach to the treatment of at least three major, devastating cancers.

One embodiment of the invention encompasses chimeric and humanized antibodies and fragments thereof capable of recognizing the AF-20 antigen associated with hepatocellular carcinoma, adenocarcinoma of the lung, colorectal carcinoma and other cancers (“AF-20 antibodies”). A preferred embodiment of this invention relates to the chimeric antibody and to the humanized antibody described herein, including the sequences of the VRs, FRs and CDRs polypeptides and the polynucleotides encoding them.

Another embodiment of the invention encompasses VR, FR and CDR polypeptides described herein in non-human AF-20 antibodies and in humanized AF-20 antibodies (“VRs, FRs and CDRs”), and the polynucleotides encoding the same, as well as the use of these polynucleotides and polypeptides in the creation of novel antibodies and polypeptide compositions capable of recognizing the AF-20 antigen.

An additional embodiment of the invention provides polynucleotides encoding AF-20 antibody polypeptides, VRs, FRs and CDRs. Various expression vectors comprising polynucleotides encoding AF-20 antibodies and VRs, FRs and CDRs operably associated with promoter sequences also are provided. Similarly, another embodiment of the invention contemplates host cells transformed with expression vectors for the expression of AF-20 antibodies, VRs, FRs and CDRs.

Embodiments of the invention also pertain to the use of AF-20 antibodies for the diagnosis, assessment and treatment of hepatocellular carcinoma, adenocarcinoma of the lung, colorectal carcinoma and other cancers which express the AF-20 antigen. Additional embodiments of the invention relate to the use of such antibodies as targeted delivery systems for cytotoxic agents such as chemotherapeutic drugs, peptides or radionuclides, for immunological response promoters such as cytokines, for pro-drugs or for gene therapies.

Another embodiment of the invention relates to the use of humanized AF-20 antibodies, and VRs, FRs and CDRs thereof, in directed molecular evolution technologies such as phage display or bacterial or yeast cell surface display technologies in order to generate polypeptides with enhanced affinity, specificity, stability or other desired characteristics.

Other objects, features, and characteristics of the present invention will become apparent upon consideration of the following description and the appended claims.

### BRIEF DESCRIPTION OF THE FIGURES

FIG. 1 shows the DNA (SEQ ID NO: 1) and amino acid sequence (SEQ ID NOS: 2 and 3, respectively, in order of appearance) of NYR-1002 V_{H} chain together with the four FRs and three CDRs identified in it.

FIG. 2 shows the DNA (SEQ ID NO: 4) and amino acid sequence (SEQ ID NOS: 5 and 6, respectively, in order of appearance) of NYR-1002 variable light chain together with the four FRs and three CDRs identified in it.

FIG. 3 shows the antibody heavy chain expression vector.

FIG. 4 shows the antibody light chain expression vector.

FIG. 5 shows the potential human T cell epitopes identified in the heavy and light variable regions of NYR-1002 (SEQ ID NOS: 7-23, respectively, in order of appearance).

FIG. 6 shows the amino acid changes and potential epitopes created in variants of V_{H} regions of NYR-1002. Table 2 discloses SEQ ID NOS: 24-31, respectively, in order of appearance.

FIG. 7 shows the amino acid changes and potential epitopes created in variants of V_{K} regions of NYR-1002. Table 2 discloses SEQ ID NOS: 32-37, respectively, in order of appearance.

FIG. 8 shows the DNA (SEQ ID NO: 38) and amino acid sequence (SEQ ID NOS: 39 and 40, respectively, in order of appearance) of primary NYDIVH1.

FIG. 9 shows the DNA (SEQ ID NO: 41) and amino acid sequence (SEQ ID NOS: 42 and 43, respectively, in order of appearance) of primary NYR-1002 V_{K} variant, NYDIVK1.

FIG. 10 shows the modified antibodies produced and yield of protein A purified antibody from 1 litre of culture supernatant.

FIG. 11 Response of 20 donors to NYDIVH2/NYDIVK2 antibody and NYR-1002 murine antibody in human T cell assays.

### DETAILED DESCRIPTION

In general, the following words or phrases have the indicated definitions when used in the description, examples, and claims:

The expressions “AF-20” or “AF-20 antigen” refer to the adenocarinoma cell antigen described in U.S. Pat. No. 5,703,213, the disclosure of which is incorporated herein by reference in its entirety, and capable of binding to the murine antibody produced by the hybridoma cell line deposited under the Budapest Treaty with the American Type Culture Collection (“ATCC”) on Apr. 12, 1988 and given the ATCC Deposit Accession No. HB 9687.

The expressions “AF-20 moAb,” “AF-20 antibody,” or “AF-20 monoclonal antibody” refer to a non-human antibody and fragments thereof that is capable of binding to the AF-20 antigen. These expressions specifically include the murine antibody described and claimed in U.S. Pat. No. 5,703,213 and produced by the hybridoma cell line deposited under the Budapest Treaty with the American Type Culture Collection (ATCC) on Apr. 12, 1988 and given the ATCC Deposit Accession No. HB 9687.

As used herein, “ATCC” shall mean the American Type Culture Collection, located at 10801 University Boulevard, Manassas, Va., 20110-2209, USA. “NYR-1002” refers to the murine antibody produced by the hybridoma cell line ATCC designation HB 9687.

The expression “constant region” or “CR” refers to the constant domains of an antibody that are not involved directly in binding the antibody to an antigen, but that are involved in various effector functions, such as participation of the antibody in antibody dependent cellular cytotoxicity.

The general structure of antibodies in vertebrates now is well understood (Edelman, G. M., Ann. N.Y. Acad. Sci., 190: 5 (1971)). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the F_{C} region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (Kabat, E. A., Structural Concepts in Immunology and Immunochemistry, 2nd Ed., p. 413-436, Holt, Rinehart, Winston (1976)), and other cellular responses (Andrews, D. W., et al., Clinical Immunobiology, pp 1-18, W. B. Sanders (1980); Kohl, S., et al., Immunology, 48: 187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

The expression “variable region” or “VR” refers to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

The expressions “complementarity determining region,” “hypervariable region,” or “CDR” refer to one or more of the hyper-variable or complementarity determining regions (CDRs) found in the variable regions of light or heavy chains of an antibody (See Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, with the CDRs from the other chain, contribute to the formation of the antigen binding site.

The expressions “framework region” or “FR” refer to one or more of the framework regions within the variable regions of the light and heavy chains of an antibody (See Kabat, E. A. et al., Sequences of Proteins of Immunological Interest, National Institutes of Health, Bethesda, Md., (1987)). These expressions include those amino acid sequences regions interposed between the CDRs within the variable regions of the light and heavy chains of an antibody.

CDR and FR residues are determined according to a standard sequence definition (Kabat et al., Sequences of Proteins of Immunological Interest, National Institutes of Health, Bethesda Md. (1987), and a structural definition (as in Chothia and Lesk, J. Mot. Biol. 196:901-217 (1987). Where these two methods result in slightly different identifications of a CDR, the structural definition is preferred, but the residues identified by the sequence definition method are considered important FR residues for determining which framework residues to import into a consensus sequence.

Throughout this description, reference is made to the numbering scheme from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. The Kabat numbering scheme is followed in this description.

For purposes of this invention, to assign residue numbers to a candidate antibody amino acid sequence which is not included in the Kabat compendium, one follows the following steps. Generally, the candidate sequence is aligned with any inmunoglobulin sequence or any consensus sequence in Kabat. Alignment may be done by hand, or by computer using commonly accepted computer programs. Alignment may be facilitated by using some amino acid residues that are common to most F_{ab} sequences. For example, the light and heavy chains each typically have two cysteines that have the same residue numbers; in V_{L} domain the two cysteines typically are at residue numbers 23 and 88, and in the V_{H} domain the two cysteine residues typically are numbered 22 and 92.

Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues. For candidate sequences which, for example, align with a Kabat sequence for residues 34 and 36 but have no residue between them to align with residue 35, the number 35 is simply not assigned to a residue.

The term “antibody” is used in the broadest sense and specifically covers single monoclonal antibodies (including agonist and antagonist antibodies) and antibody compositions with polyepitopic specificity. The term “antibody” also includes obvious variants, derivatives, analogs, fragments, mimetics, all of which substantially retain the binding characteristics and other properties of the stated antibody.

The expression “monoclonal antibody” (moAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single epitopic region of an antigen. Furthermore, in contrast to conventional (polyclonal) antibody preparations that typically include different antibodies directed against different determinants (epitopes), each moAb is directed against a single determinant on the antigen. In addition to their specificity, monoclonal antibodies are advantageous in that they can be synthesized by hybridoma culture, uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by the hybridoma method first described by Kohler et al., Nature, 256:495 (1975), or may be made by recombinant DNA methods (see, e.g., U.S. Pat. No. 4,816,567 to Cabilly et al.). The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991), for example.

“Antibody fragment,” and all grammatical variants thereof, as used herein are defined as a portion of an intact antibody comprising the antigen binding site or variable region of the intact antibody, wherein the portion is free of the constant heavy chain domains (i.e., CH2, CH3, and CH4, depending on antibody isotype) of the F_{C} region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; and (3) single chain polypeptides containing only one heavy chain variable region, or a fragment thereof containing the three CDRs of the heavy chain variable region, without an associated light chain moiety. Antibody fragments of the invention further encompass multispecific or multivalent structures formed from the aforementioned antibody fragments. In an antibody fragment comprising one or more heavy chains, the heavy chain(s) can contain any one of the following:
one or more constant domain sequences (e.g., CH1 in the IgG isotype) found in a non-Fc region of an intact antibody, and/or
any hinge region sequence found in an intact antibody, and/or
a leucine zipper sequence fused to or situated in the hinge region sequence or the constant domain sequence of the heavy chain(s). Suitable leucine zipper sequences include the jun and fos leucine zippers taught by Kostelney et al., J. Immunol., 148: 1547-1553 (1992) and the GCN4 leucine zipper described in the examples below.

The expression “chimeric antibody” refers to a polypeptide comprising the variable region of a non-human antibody that binds AF-20 linked to at least another part of another protein, preferably the constant region of a human antibody.

The term “humanized” insofar as it pertains to a “humanized” antibody refers to a polypeptide comprising a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which some or all CDR residues and/or possibly some FR residues are substituted by residues from analogous sites in rodent or other non-human antibodies that are capable of binding to the AF-20 antigen. The expression “humanized antibody” also includes an immunoglobulin amino acid sequence variant or fragment thereof that is capable of binding to the AF-20 antigen and that comprises a FR region having substantially the amino acid sequence of a human immunoglobulin and a CDR having substantially the amino acid sequence of a non-human immunoglobulin.

In general, the humanized antibody will comprise substantially all of at least one, and more preferably two, variable domains (Fab, Fab′, F(ab′)₂, Fabc, Fv) in which all or substantially all of the CDR regions correspond to those of a non-human inmunoglobulin, and all or substantially all of the FR regions are those of a human immunoglobulin consensus sequence. The humanized antibody optimally also will comprise at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody may be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG and preferably IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The FR and CDR regions of the humanized antibody need not correspond precisely to the parental sequences, e.g., the import CDR or the consensus FR may be mutagenized by substitution, insertion or deletion of one or more residues so that the CDR or FR residue at that site does not correspond to either the consensus or the import antibody. Such mutations, however, will not be extensive and will not dramatically affect binding of the antibody to the binding target.

The expression “humanized antibody” also includes hybrid and recombinant antibodies and polypeptides produced by splicing a variable region or one or more CDRs of an anti-AF-20 antibody with any heterologous protein(s), regardless of species of origin, type of protein, immunoglobulin class or subclass designation, so long as the hybrid and recombinant antibodies and polypeptides exhibit the desired biological activity.

The expression “humanized antibody” further includes antibodies and polypeptides rendered non-immunogenic, or having reduced immunogenicity relative to the native antibody, to a human by the method of determining at least part of the amino acid sequence of the antibody or polypeptide (preferably that part of non-human origin such as a V_{H} or V_{K} region of a non-human antibody), identifying in the amino acid sequence one or more potential epitopes for human T-cells, and modifying the amino acid sequence(s) of the one or more epitopes to eliminate at least one of the T-cell epitopes identified in order to eliminate or reduce the immunogenicity of the protein or portions thereof when exposed to the human immune system. In the creation of a humanized antibody, preferably about 75%, more preferably about 90%, and most preferably greater than about 95% of the humanized antibody residues will correspond to those of the parental FR and CDR sequences.

The phrase “T-cell epitopes” refers to specific peptide sequences that either bind with reasonable efficiency to MHC class II molecules or which, from previous or other studies, show the ability to stimulate T-cells via presentation on MHC class II molecules. It will be understood, however, that not all such peptide sequences will be delivered into the correct MHC class II cellular compartment for MHC class II binding or will be suitably released from a larger cellular protein for subsequent MHC class II binding. It also will be understood that even such peptides that are presented by MHC class II on the surface of antigen-presenting cells will elicit a T cell response for reasons including a lack of appropriate T cell specificity and tolerance by the immune system to the particular peptide sequence.

The expression “bifunctional antibody” refers to an antibody that may have one aim having a specificity for one antigenic site, such as a tumor associated antigen, while the other arm recognizes a different target, for example, a hapten which is, or to that is bound, an agent lethal to the antigen-bearing tumor cell. Alternatively, the bifunctional antibody may be one in which each aim has specificity for a different epitope of a tumor associated antigen of the cell to be therapeutically or biologically modified. In any case, the hybrid antibodies have a dual specificity, preferably with one or more binding sites specific for the hapten of choice or one or more binding sites specific for a target antigen, for example, an antigen associated with a tumor, an infectious organism, or other disease state.

Biological bifunctional antibodies are described, for example, in European Patent Application EPA 0 105 360, to which those skilled in the art are referred. Such hybrid or bifunctional antibodies may be derived, as noted, either biologically by cell fusion techniques, or chemically, especially with cross-linking agents or disulfide bridge-forming reagents, and may be comprised of those antibodies and/or fragments thereof. Methods for obtaining such hybrid antibodies are disclosed, for example, in International Publication No. W083/03679, published Oct. 27, 1983, and European Patent Application EPA 0 217 577, published Apr. 8, 1987, the disclosures of both of which are incorporated herein by reference in their entireties. Particularly preferred bifunctional antibodies are those biologically prepared from a “polydome” or “quadroma” or which are synthetically prepared with cross-linking agents such as bis-(maleimideo)-methyl ether (“BMME”), or with other cross-linking agents familiar to those skilled in the art.

The term “conjugated” means to couple directly or indirectly one molecule with another by numerous means, e.g., by covalent bonding, by non-covalent bonding, by ionic bonding, or by non-ionic bonding. Covalent bonding includes bonding by various linkers such as thioether linkers or thioester linkers. Direct coupling involves one molecule attached to another molecule of interest. Indirect coupling involves one molecule attached to another molecule not of interest which acts as a bridge and in turn is attached directly or indirectly to the molecule of interest.

The expression “cytotoxic agent” means any agent that is detrimental to cells. Examples include antimetabolites such as methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

In a particularly preferred embodiment, cytotoxic agents include one or more of the NTP peptides disclosed in U.S. application Ser. No. 10/153,334 which is now U.S. Pat. No. 6,924,266, U.S. application Ser. No. 10/198,070 which is now U.S. Pat. No. 7,241,738, U.S. application Ser. No. 10/198,069 which is now U.S. Pat. No. 7,192,929, and U.S. application Ser. No. 10/294,891, the disclosures of each of which are incorporated by reference herein in their entirety. These NTP peptide, NTP peptide fragments, and the like, may be conjugated to the humanized antibodies described herein to facilitate tumor cell necrosis.

“Oligonucleotides” as used herein denotes short-length, single- or double-stranded deoxynucleotides that are chemically synthesized by known methods (such as phosphotriester, phosphate, or phosphoramidite chemistry, using solid phase techniques such as described in EP 266,032 published May 4, 1988, or via deoxynucleoside H-phosphonate intermediates as described by Froehler et al., Nucl. Acids Res., 14: 5399-2407 (1986)). They are then purified on polyacrylamide gels.

The technique of “polymerase chain reaction,” or “PCR,” as used herein, generally refers to a procedure wherein minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; the primers being identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N.Y., 1989). As used herein, PCR is considered to be one, but not the only, example of a nucleic acid polymerase reaction method for amplifying a nucleic acid test sample, comprising the use of a known nucleic acid (DNA or RNA) as a primer and utilizing a nucleic acid polymerase to amplify or generate a specific piece of nucleic acid or to amplify or generate a specific piece of nucleic acid which is complementary to a particular nucleic acid.

“Treatment” as it is used herein refers to both therapeutic treatment and prophylactic or preventative measures. Those in need of treatment include those already with the disorder as well as those prone to have the disorder or those in which the disorder is to be prevented.

Features of embodiments described herein relate to humanized and chimeric antibodies, fragments, polypeptides or derivatives thereof that are capable of binding to adenocarcinoma cell antigen AF-20, which is associated with carcinoma cells, and especially with hepatocarcinoma cells, and adenocarcinoma cells of the colon and lung. More specifically, embodiments relate to humanized and chimeric antibodies, fragments, polyp eptides or derivatives thereof derived from a murine monoclonal antibody that is produced by hybridoma cell line ATCC designation HB 9687 or other non-human antibodies that specifically bind AF-20. The embodiments described herein also relate to nucleic acid sequences that express the present invention's humanized and chimeric antibodies, fragments, polypeptides or derivatives thereof, methods for producing such humanized and chimeric antibodies, fragments, polypeptides and derivatives specific to AF-20, methods for using such humanized and chimeric antibodies, fragments, polypeptides and derivatives to generate other polypeptides, variants and derivatives specific to AF-20, continuous hybridoma cell lines capable of secreting such humanized and chimeric antibodies, pharmaceutical and diagnostic compositions containing such humanized or chimeric antibodies or fragments or derivatives thereof, and methods of use thereof for the treatment or diagnosis of cancer.

Various embodiments described herein arise from the creation of chimeric and humanized antibodies from the murine AF-20 antibody that are capable of binding to AF-20. Surprisingly, the chimeric antibody, chNYR-1002, and the humanized antibody, huNYR-1002, proved capable of binding to the AF-20 antigen.

One embodiment of this invention provides a chimeric derivative of the AF-20 antibody, chNYR-1002, in which the murine variable regions of NYR-1002, V_{H} and V_{K}, were joined onto human IgG1 or K constant regions respectively. Other embodiments include other chimeric derivatives of NYR-1002 having different human constant regions, such as IgG2 or lambda constant regions that are used to join the murine variable regions of NYR-1002, V_{H} and V_{K}.

Another embodiment provides a humanized derivative of the chNYR-1002 chimeric antibody, huNYR-1002, in which potential human T-cell epitopes have been identified (in the amino acid sequences of the V_{H} and V_{K} regions of chNYR-1002), and the amino acid sequences of the putative T-cell epitopes modified to eliminate one or more of the T-cell epitopes identified to eliminate or reduce the immunogenicity of the antibody. The embodiments also encompasses other humanized derivatives of NYR-1002 in which different human constant regions, such as IgG2 or lambda constant regions are used to join with the humanized variable regions of NYR-1002, V_{H} and V_{K}, contained in huNYR-1002.

A further embodiment provides the amino acid and corresponding nucleic acid sequences of the CDRs identified in the V_{H} and V_{K} regions of NYR-1002: CDR 1 (SEQ ID No. 44), CDR 2 (SEQ ID No. 45) and CDR 3 (SEQ ID No. 46) of the light chain and CDR 1 (SEQ ID No. 47), CDR 2 (SEQ ID No. 48) and CDR 3 (SEQ ID No. 49) of the heavy chain. The amino acid sequences of these CDRs may be modified, however. The amino acid sequence of each CDR may preferably be changed by up to 10% by amino acid substitutions, insertions and/or deletions, more preferably up to 20%, more preferably up to 30% and even more preferably up to 40%, provided that the resulting humanized antibodies comprising the amino acid sequences maintain their binding specificity for the binding target. Each CDR may therefore include one, two or more amino acid substitutions, insertions and/or deletions. Preferably the amino acid sequence of each CDR is substantially homologous to that of a specific CDR disclosed in this invention. Skilled artisans will appreciate that the listing of a specific amino acid sequence will inherently include within its listing all of these modifications, so long as the activity and utility of the sequence is substantially retained.

The polynucleotide and polypeptide sequences provided herein have utility in the generation of other humanized antibodies, for example by replacing the CDR regions of the variable regions of a human antibody. These sequences also have utility in the generation of other humanized variable regions, such as by replacing the CDR regions in the variable regions of a human antibody, where the human variable regions share significant homology with the murine variable regions of NYR-1002. These sequences also have utility in the creation of polypeptides capable of binding to the AF-20 antigen. The polynucleotide and polypeptide sequences provided herein also have utility in the identification of significantly homologous CDRs of human or humanized antibodies, such as those contained in a library or bank of such antibodies. The embodiments further encompass humanized antibodies and antibody fragments in which one or more of the original CDRs in the humanized antibody are replaced by a CDR(s) described herein. The embodiments also encompasses bispecific antibodies, antibody fragments and polypeptides containing one or more of the CDRs of this invention.

Embodiments described herein further include variants and equivalents that are substantially homologous to the humanized antibodies, antibody fragments, polypeptides, variable regions and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

The phrase “substantially homologous” is used herein in regard to the similarity of a subject amino acid sequence (of an oligo- or poly-peptide or protein) to a related, reference amino acid sequence. This phrase typically is defined as at least about 75% “correspondence,”—i.e., the state of identical amino acid residues being situated in parallel—between the subject and reference sequences when those sequences are in “alignment,” (i.e. when a minimal number of “null” bases have been inserted in the subject and/or reference sequences so as to maximize the number of existing bases in correspondence between the sequences). “Null” bases are not part of the subject and reference sequences; also, the minimal number of “null” bases inserted in the subject sequence may differ from the minimal number inserted in the reference sequence. In this definition, a reference sequence is considered “related” to a subject sequence where both amino acid sequences make up proteins or portions of proteins which are AF-20 antibodies, antibody fragments, or polypeptides with the capability of binding to AF-20. Each of the proteins comprising these AF-20 antibodies, antibody fragments or polypeptides may independently be antibodies, antibody fragments, polypeptides or bi- or multi-functional proteins, e.g., such as fusion proteins, bi- and multi-specific antibodies, single chain antibodies, or multimers thereof and the like.

A further aspect of this invention provides the amino acid and corresponding nucleic acid sequences of the humanized V_{H} and V_{K} regions of huNYR-1002. These sequences have utility in the generation of other humanized antibodies, for example as replacements for the corresponding variable regions of a human antibody. These sequences also have utility in the identification of significantly homologous variable regions of human or humanized antibodies, such as those contained in a library or bank of such antibodies. These sequences also have utility in the creation of antibody fragments and polypeptides that are capable of binding to the AF-20 antigen. The embodiments further encompass humanized antibodies and antibody fragments in which one or more of the original variable regions in the humanized antibody are replaced by a variable region(s) described herein. The embodiments also encompass bispecific antibodies, antibody fragments and polypeptides containing one or more of the humanized variable regions described herein.

Embodiments of the invention also encompass the use of the humanized antibodies, antibody fragments, CDRs and humanized variable regions described herein in directed molecular evolution technologies such as phage display technologies, and bacterial and yeast cell surface display technologies. Phage display technology (McCafferty et al., Nature 348:552 (1990)) can be used to produce novel human antibodies and antibody fragments in vitro, from variable region genes or genes encoding humanized antibodies or antibody fragments. According to this technique, antibody variable region genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell.

Phage display can be performed in a variety of formats; and for their review, See, e.g., Johnson et al., Current Opinion in Structural Biology 3:564 (1993). Variable gene segments can be used for phage display. Clackson et al., (Nature 352:624 (1991)) isolated a diverse array of anti-oxazolone antibodies from a small random combinatorial library of variable region genes derived from the spleens of immunized mice. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing techniques that introduce small random mutations in the antibody genes. In this method, the affinity, specificity, immunogenicity or other characteristics of humanized antibodies can be improved and new humanized antibodies, antibody fragments and polypeptides capable of binding to the AF-20 antigen be discovered.

Another embodiment encompasses chimeric and humanized antibodies and antibody fragments derived from other non-human monoclonal antibodies that bind AF-20. The raising of monoclonal antibodies against a desired antigen is well known in the art. U.S. Pat. No. 5,703,213 describes one method of generating murine monoclonal antibodies to the AF-20 antigen by immunizing mice with FOCUS HCC cells. This method is applicable to the generation of other AF-20 antibodies from mice and other non-human host animals.

Other methods to generate non-human antibodies are well-known in the art and can be applied to immunization by FOCUS HCC cells or by the AF-20 antigen itself when and if isolated. Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized by multiple subcutaneous (sc) or intraperitoneal (ip) injections of antigen and an adjuvant, such as monophosphoryl lipid A (MPL)/trehalose dicrynomycolate (TDM) (Ribi Immunochem. Research, Inc., Hamilton, Mont.), at multiple sites. Two weeks later, the animals are boosted, and 7 to 14 days later animals are bled and the serum is assayed for anti-antigen titer. Animals are boosted until titer plateaus. Sera are harvested from animals, and polyclonal antibodies are isolated from sera by conventional immunoglobulin purification procedures, such as protein A-Sepharose chromatography, hydroxylapatite chromatography, gel filtration, dialysis, or antigen affinity chromatography.

The method described above is used to elicit lymphocytes that produce or are capable of producing antibodies that will specifically bind to the immunizing agent. Alternatively, lymphocytes may be immunized in vitro. Lymphocytes then can be fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared can be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the ATCC. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing can be assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson et al., Anal. Biochem., 107:220 (1980). One such method of determining binding specificity to the AF-20 antigen is described in U.S. Pat. No. 5,703,213.

Antibodies of embodiments of the invention also may be described or specified in terms of their binding affinity to an AF-20 polypeptide. Preferred binding affinities include those with a dissociation constant or Kd less than 1 μM, more preferably less than about 100 nM, and most preferably less than about 1 nM.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones can be suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies can readily be isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs., 130: 151 (1992). Other methods to generate non-human monoclonal antibodies that are capable of binding to the AF-20 antigen also exist.

The monoclonal antibodies thus produced and the DNA encoding such antibodies can then be used to produce chimeric antibodies, humanized antibodies and antibody fragments in accordance with the methods described in the invention or with other methods known to those skilled in the art.

A preferred method is to render the non-human antibody non-immunogenic or less immunogenic to a human by determining at least part of the amino acid sequence of the antibody (preferably that part of non-human origin such as a V_{H} or V_{K} region of a non-human antibody), of one or more potential epitopes for human T-cells, and modifying the amino acid sequence to eliminate at least one of the putative T-cell epitopes, thereby eliminating or reducing the immunogenicity of the protein or a part thereof when exposed to the human immune system. Following these methods, a panel of modified antibodies may be generated. The resulting modified antibodies then can be screened for expression level, immunogenicity and affinity and specificity for the AF-20 antigen and the best candidate(s) selected.

Other methods of generating chimeric antibodies and humanized antibodies from non-human antibodies or of reducing the immunogenicity of non-human antibodies are known to those having ordinary skill in the art, and include but are not limited to:
the creation of chimeric antibodies by attaching the variable regions in the heavy and light chains of the non-human antibody onto the constant regions of a human antibody as described by Cabilly, et al. in U.S. Pat. No. 4,816,567; Morrison, S. L. et al., Proc. Natl. Acad. Sci. USA 81:6851-6855 (1984); Boulianne, G. L. et al., Nature 312;643-646 (1984); Neuberger, M. S. et al., Nature 314:268-270 (1985).
the creation of humanized antibodies by the substitution of non-human complementarity determining regions (CDRs) or CDR sequences for the corresponding segments of a human antibody as described by Winter in U.S. Pat. No. 5,225,539, and in Jones, P. T. et al., Nature 321:522-525 (1986); Riechmann, L. et al, Nature 332:323-327 (1988); Verhoeyen, M. et al., Science 239:1534-1536 (1988). This may also entail the substitution of some FR residues in the human antibody with residues from analogous sites in non-human antibody in order to retain antigen binding as described, for example, by Queen, et al. in U.S. Pat. Nos. 5,530,101, 5,585,089, 5,693,762, 6,180,370 Carter, et al. in U.S. Pat. Nos. 6,054,297, 6,407,213 and 6,639,055, Adair in U.S. Pat. No. 6,632,927 and Winter in U.S. Pat. No. 6,548,640.
the creation of humanized antibodies by selective substitution of residues in the variable regions of the non-human antibody as described, for example, by Pedersen, et al. in U.S. Pat. No. 5,639,641, Studnicka, et al. in U.S. Pat. Nos. 5,766,886 and 5,821,123, and Carr et al, in U.S. patent application Ser. No. 10/300215, now U.S. Pat. No. 7,125,689.
the linking of an antibody or antibody fragment to auto-antigenic sequences which render the non-human antibody or antibody fragment less immunogenic as described by Jordan, et al. in U.S. Pat. No. 6,652,863.

As described above, DNA encoding the monoclonal antibody or antibody fragment of interest can be isolated from its hybridoma or phage display clone of origin, and then manipulated to create humanized and/or affinity matured constructs. In addition, known techniques can be employed to introduce an amino acid residue or residues into any desired location on the polypeptide backbone of the antibody fragment, e.g., a cysteine residue placed in the hinge region of the heavy chain, thereby providing a site for specific attachment of polymer molecule(s). In one embodiment, the native cysteine residue in either the light or heavy chain of the antibody fragment that ordinarily forms the disulfide bridge linking the light and heavy chains is substituted with another amino acid, such as serine, in order to leave the partner cysteine residue in the opposite chain with a free suflhydryl for specific attachment of a polymer molecule.

Upon construction of the desired antibody or antibody fragment-encoding clone, the clone can be used for recombinant production of the antibody or antibody fragment using methods known to those skilled in the arts. Finally, the antibody or antibody fragment product can be recovered from host cell culture and purified using methods known to those skilled in the art or described herein. In the case of embodiments utilizing an antibody fragment engineered to lack a cysteine residue as described supra, preferred recombinant production systems include bacterial expression and product recovery procedures known to those skilled in the art or described herein. If a full length antibody is produced, the desired antibody fragment can be obtained therefrom by subjecting the intact antibody to enzymatic digestion according to methods known in the art.

The chimeric and humanized antibodies, fragments and polypeptides of the embodiments may be made according to known methods. One detailed method for production is set forth in the Examples. It should be understood that one of ordinary skill in the art will be able to substitute known conventional techniques for those described below for the purpose of achieving the same or similar results. The humanized antibodies of the embodiments described herein may be produced by the following process:

(a) constructing, by conventional techniques, an expression vector containing (1) an operon with (2) a DNA sequence encoding an antibody heavy chain in which the CDRs and such minimal portions of the variable domain framework region that are required to retain antibody binding specificity are derived from a non-human antibody, and (3) the remaining parts of the antibody chain derived from a human antibody, thereby producing the vector of the invention;

(b) constructing, by conventional techniques, an expression vector containing an operon with a DNA sequence encoding a complementary antibody light chain in which the CDRs and such minimal portions of the variable domain framework region that are required to retain donor antibody binding specificity are derived from a non-human antibody, and the remaining parts of the antibody chain are derived from a human antibody, thereby producing the vector of the invention;

(c) transfecting the expression vectors into a host cell by conventional techniques to produce the transfected host cell of the invention; and

(d) culturing the transfected host cell by conventional techniques to produce the altered antibody of the invention.

The host cell may be cotransfected with the two vectors of the invention, the first vector containing an operon encoding a light chain derived polypeptide and the second vector containing an operon encoding a heavy chain derived polypeptide. The two vectors contain different selectable markers, but apart from the antibody heavy and light chain coding sequences, are preferably identical, to desirably achieve equal expression of the heavy and light chain polyp eptides. Alternatively, a single vector may be used, the vector including the sequences encoding both the light and the heavy chain polypeptides. The coding sequences for the light and heavy chains may comprise cDNA, genomic DNA, or both.

The host cell used to express the altered antibody of the invention may be either a bacterial cell such as Escherichia coli, or a eukaryotic cell. In particularly preferred embodiments of the invention, a mammalian cell of a well defined type for this purpose, such as a myeloma cell or a Chinese hamster ovary (CHO) cell may be used.

The general methods by which the vectors of the invention may be constructed, transfection methods required to produce the host cell of the invention and culture methods required to produce the antibody of the invention from such host cells all include conventional techniques. The Examples below provide one such method. Although preferably the cell line used to produce the humanized antibody is a mammalian cell line, any other suitable cell line, such as a bacterial cell line or a yeast cell line, may alternatively be used. In particular, it is envisaged that E. coli-derived bacterial strains could be used.

Likewise, once produced the humanized antibodies of embodiments of the invention may be purified according to standard procedures of the art, including cross-flow filtration, ammonium sulphate precipitation, affinity column chromatography, gel electrophoresis and the like.

It should be understood that the humanized antibodies of the embodiments perform in a manner identical or substantially similar to that of non-humanized versions of the same antibodies. Preferably, however, the humanized antibodies are more advantageously used in a human, when compared to the non-humanized versions of the same antibodies. The humanized antibodies of the embodiments may be used for the design and synthesis of either peptide or non-peptide compounds (mimetics) that would be useful for the same therapy as the antibody (Saragobi et al., Science 253:792-795 (1991)), the contents of which is herein incorporated by reference in its entirety.

Embodiments of the invention also encompass fragments of humanized antibodies capable of binding to the AF-20 antigen. Antibody fragments can provide significant advantages over intact antibodies, notably the fact that recombinant antibody fragments can be made in bacterial cell expression systems. Bacterial cell expression systems provide several advantages over mammalian cell expression systems, including reduced time and cost at both the research and development and manufacturing stages of a product.

Antibody fragments can be produced by any method known in the art or described herein. Generally, an antibody fragment is derived from a parental intact antibody. The desired antibody fragments can be generated from purified antibody preparations by conventional enzymatic methods, e.g. F(ab′)₂ fragments are produced by pepsin cleavage of intact antibody, and Fab fragments are produced by briefly digesting intact antibody with papain.

Certain embodiments also include the use of bispecific and heteroconjugate antibody fragments having specificities for at least two different antigens. Bispecific and heteroconjugate antibodies can be prepared as full length antibodies or as antibody fragments (e.g. F(ab′)₂ bispecific antibody fragments). Antibody fragments having more than two valencies (e.g. trivalent or higher valency antibody fragments) also are contemplated for use herein. Bispecific antibodies, heteroconjugate antibodies, and multi-valent antibodies can be prepared by methods known to those skilled in the art or described herein.

The embodiments of the invention also include therapeutic compositions containing the humanized antibodies, antibody fragments and polypeptides described herein. For example, the humanized antibodies, antibody fragments and polypeptides described herein may be conjugated to an effector moiety having therapeutic activity and used to selectively target cells that express the AF-20 antigen. Such conjugates would take advantage of the internalization of AF-20 antibodies upon binding to the AF-20 antigen. Many such effector moieties are known in the art and include cytotoxic agents, immunological response modifiers, oligonucleotides, genes, viral vectors containing therapeutic genes, liposomes containing genes or cytotoxic agents, or prodrugs or enzymes.

Many cytotoxic agents are known to those skilled in the arts. These include chemotherapeutic agents such as carboplatin, cisplatin, paclitaxel, gemcitabine, calicheamicin, doxorubicin, 5-fluorouracil, mitomycin C, actinomycin D, cyclophosphamide, vincristine and bleomycin. Toxic enzymes from plants and bacteria such as ricin, diphtheria toxin and Pseudomonas toxin may be conjugated to the humanized antibodies, antibody fragments and polypeptides of this invention to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)). Other cytotoxic agents include cytotoxic ribonucleases as described by Goldenberg in U.S. Pat. No. 6,653,104.

Embodiments of the invention also relate to radioimmunoconjugates where a radionuclide that emits alpha or beta particles is stably coupled to the antibody, antibody fragment or polypeptide with or without the use of a complex-forming agent. Such radionuclides include beta-emitters such as Phosphorus-32 (³²P), Scandium-47 (⁴⁷Sc), Copper-67 (⁶⁷Cu), Gallium-67 (⁶⁷Ga), Yttrium-88 (⁸⁸Y), Yttrium-90 (⁹⁰Y), Iodine-125 (¹²⁵I), Iodine-131 (¹³¹I), Samarium-153 (¹⁵³Sm), Lutetium-177(¹⁷⁷Lu), Rhenium-186 (¹⁸⁶Re) or Rhenium-188 (¹⁸⁸Re), and alpha-emitters such as Astatine-211 (²¹¹ At), Lead-212 (²¹²Pb), Bismuth-212 (²¹²Bi) or -213 (²¹³Bi) or Actinium-225 (²²⁵Ac).

A particularly preferred embodiment comprises the conjugation of NTP peptides or derivatives thereof to humanized antibodies, and should be contemplated as useful in the embodiments.

The therapeutic compositions may be used to introduce immunological response modifiers into tumor cells that express the AF-20 antigen and thereby either directly or indirectly mark the tumor cells for destruction by the patient's immune system. The therapeutic compositions also may be used to introduce gene sequences into tumor cells that express the AF-20 antigen and thereby enable the expression of the gene in the tumor cells. The gene may replace or supplement genes, the functioning of which in the tumor cell is either impaired or non-existent, thereby inducing cell death through apoptosis or other mechanisms, inhibiting or preventing tumor cell proliferation or migration, marking the tumor cells for destruction by the patient's immune system, or having similar or other therapeutic effects. The gene may be exogenous to the tumor cell genome. Such a gene may express a cytotoxic protein or an enzyme capable of cleaving a prodrug into a cytotoxic moiety. Such gene sequences may be delivered to the target cell(s) by means of gene delivery system, such as viral vectors or liposomes, conjugated to the therapeutic compositions of this invention.

Similarly, the therapeutic compositions may be used to introduce oligonucleotides into tumor cells that express the AF-20 antigen. Such oligonucleotides may include antisense oligonucleotides that inhibit the functioning of targeted mRNA in the tumor cell; short interfering ribonucleic acids (siRNAs) that inhibit the expression of proteins in the tumor cell necessary for viability, proliferation or migration; ribozymes; agents that increase the susceptibility of the tumor cell to other anti-cancer treatments; and triple helix-forming oligonucleotides.

The embodiments also contemplate pharmaceutical compositions containing different therapeutic compositions, such as two or more different antibody conjugates, each with either a different antibody or with a different effector moiety. The effector moiety may include genes or other oligonucleotides which, when introduced into a tumor cell as a result of the internalization of the antibody therapeutic, provide a favorable therapeutic response such as inducing cellular apoptosis; replacing a dysfunctional gene; expression of a therapeutically favorable protein; and the like. The gene may be enclosed in a liposome or attached to an appropriate vector such as a virus.

In another embodiment of the invention, the specificity for AF-20 and the reduced immunogenicity render the chimeric and humanized antibodies described herein suitable for use as diagnostic agents when conjugated to a detectable label for the detection of different cancer types such as hepaocellular carcinomas, adenocarcinomas of the lung and colorectal carcinomas. Such compositions may be useful for the diagnosis, assessment of the appropriate treatment and evaluation of the prognosis of cancers characterized by AF-20 expression (based on levels of AF-20 expression); they may be useful as tumor imaging agents, or useful as radiolabeled antibodies in the Radioimmunoguided Surgery.RTM. System (RIGS.RTM.). See Hinkle et al., Antibody, Immunoconjugates and Radiopharmaceuticals, 4(3):339-358 (1991).

Many detectable labels that may be conjugated to an antibody or polypeptide are known in the arts. Detectable labels include radionuclides such as ³H, ¹¹C, ¹⁴C, ¹⁸F, ⁶⁴Cu, ⁷⁶Br, ⁸⁶Y, ^{99m}Tc, ¹¹¹In, ¹²³I, ¹²⁵I, or ¹⁷⁷Lu. Methods of detecting such labels include PET scans and immunoscintigraphy. Detectable labels for in vitro assays include enzymes such as horseradish peroxidase; fluorophores; chromophores; chemiluminescent agents; radionuclides; chelating complexes; dyes; colloidal gold or latex particles.

The diagnostic compositions of this embodiment also may be used in in vitro assays to determine whether a person or animal has a cancer that expresses the AF-20 antigen. Such assays would have use in cancer diagnosis, staging and treatment assessment. Preferably such assays would be used to determine whether the patient or animal has a cancer that is susceptible to treatment with a therapeutic composition capable of binding to tumor cells expressing the AF-20 antigen. Most preferably such assays would be used to determine whether and how to treat a patient or animal with a therapeutic composition of this invention.

Methods of developing such assays are known in the art. Assay types include, but are not limited to, immunohistological assays of biopsied tissue with a diagnostic composition of this invention and immunoassays where a sample of tissue or bodily fluid is contacted with a diagnostic composition of this invention.

Many methods of conjugating effector moieties or detectable labels are known to those skilled in the art. The attachment of antibodies to desired effectors is well known. See, e.g., U.S. Pat. No. 5,435,990 to Cheng et al., the disclosure of which is incorporated herein by reference in its entirety. Moreover, bifunctional linkers for facilitating such attachment are well known in the art and widely available. Also, chelators (chelants and chelates) providing for attachment of radionuclides are well known in the art and are readily available.

The therapeutic and diagnostic compositions may have utility in the diagnosis and treatment of cancers. Preferably such cancers are adenocarcinomas, and most preferably such cancers are hepatocellular carcinomas, adenocarcinomas of the lung and colorectal carcinomas.

The therapeutic compositions have further utility in the treatment of cancers and other tumors that express the AF-20 antigen. They may be used alone or in conjunction with other anti-cancer or anti-tumor treatments such as chemotherapy, immunotherapy, radiation, surgical excision or ablative therapies.

One skilled in the art will be capable (by routine experimentation) of determining the amount of antibody, antibody fragment or polypeptide that would be effective and non-toxic for the purpose of treating a particular cancer. Generally, however, an effective dosage will be in the range of about 0.05 to 100 milligrams per kilogram body weight per day, and preferably from about 0.5 to about 25 milligrams per kilogram body weight per day.

The chimeric or humanized antibodies, antibody fragments or polypeptides described herein may be administered to a human or other animal in accordance with the aforementioned methods of treatment in an amount sufficient to produce a therapeutic or prophylactic effect. Antibodies may be administered to such human or other animal in a conventional dosage form prepared by combining the antibody with a conventional, pharmaceutically acceptable carrier, diluent, and/or excipient, according to known techniques. It will be recognized by one of ordinary skill in the art that the form and character of the pharmaceutically acceptable carrier, diluent, and/or excipient is dictated by the amount of active ingredient with which it is to be combined, the route of administration, and other well-known variables.

Pharmaceutically acceptable formulations may include, e.g., a suitable solvent, preservatives such as benzyl alcohol if desired, and a buffer. Useful solvent may include, e.g., water, aqueous alcohols, glycols, and phosophonate and carbonate esters. Such aqueous solutions contain no more than 50% by volume of organic solvent. Suspension-type formulations may include a liquid suspending medium as a carrier, e.g., aqueous polyvinylpyrrolidone, inert oils such as vegetable oils or highly refined mineral oils, or aqueous cellulose ethers such as aqueous carboxymethylcellulose. A thickener such as gelatin or an alginate also may be present, one or more natural or synthetic surfactants or antifoam agents may be used, and one or more suspending agents such as sorbitol or another sugar may be employed therein. Such formations may contain one or more adjuvants.

The route of administration of the antibodies, fragment or polypeptides of the present invention may be oral, parenteral, by inhalation, or topical. The term “parenteral” as used herein includes intrathrombotic, intravenous, intramuscular, subcutaneous, rectal, vaginal, or intraperitoneal administration. The intrathrombotic, intravenous, and intramuscular forms of parenteral administration are preferred routes of administration.

The daily parenteral and oral dosage regimens for prophylactically or therapeutically employing humanized antibodies of the present invention will generally be in the range of about 0.005 to 100, but preferably about 0.5 to 10, milligrams per kilogram body weight per day.

The antibodies also may be administered by inhalation. “Inhalation” denotes intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 0.1 to about 100, more preferably about 10 to 100, milligrams per kg body weight.

The antibody also may be administered topically. Topical administration denotes non-systemic administration. This includes the administration of a humanized antibody (or humanized antibody fragment) formulation externally to the epidermis or to the buccal cavity, and instillation of such an antibody into the ear, eye, or nose, and wherever it does not significantly enter the bloodstream. Systemic administration denotes oral, intravenous, intraperitoneal, subcutaneous, and intramuscular administration. The amount of an antibody required for therapeutic, prophylactic, or diagnostic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

While it is possible for an antibody, fragment or polypeptide described herein to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0. 1% to 1% w/w of the formulation.

The topical formulations can comprise an active ingredient together with one or more acceptable carrier(s) therefor and optionally any other therapeutic ingredients(s). The carrier(s) typically is “acceptable” in the sense of being compatible with the other ingredients of the formulation and not deleterious to the recipient thereof. Formulations suitable for topical administration include liquid or semi-liquid preparations suitable for penetration through the skin to the site of where treatment is required, such as liniments, lotions, creams, ointments or pastes, and drops suitable for administration to the eye, ear, or nose.

Drops may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes typically are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active agent such as sorbitan esters or polyoxyethylene derivatives thereof. Suspending agents such as natural gums, cellulose derivatives or inorganic materials such as silicaceous silicas, and other ingredients such as lanolin, may also be included.

Kits according to an embodiment include frozen or lyophilized chimeric or humanized antibodies, antibody fragments or polypeptide to be reconstituted, respectively, by thawing (optionally followed by further dilution) or by suspension in a (preferably buffered) liquid vehicle. The kits also may include buffer and/or excipient solutions (in liquid or frozen form)—or buffer and/or excipient powder preparations to be reconstituted with water—for the purpose of mixing with the humanized antibodies or humanized antibody fragments to produce a formulation suitable for administration. Thus, the kits containing the chimeric or humanized antibodies, antibody fragments or polypeptides preferably are frozen, lyophilized, pre-diluted, or pre-mixed at such a concentration that the addition of a predetermined amount of heat, of water, or of a solution provided in the kit will result in a formulation of sufficient concentration and pH as to be effective for in vivo or in vitro use in the treatment or diagnosis of cancer.

Preferably, such a kit also will comprise instructions for reconstituting and using the chimeric or humanized antibody, antibody fragment or polypeptide composition to treat or detect cancer. The kit also may comprise two or more component parts for the reconstituted active composition. For example, a second component part—in addition to tie chimeric or humanized antibodies, antibody fragments or polypeptides—may be a bifunctional chelant, a bifunctional chelate, or a therapeutic agent such as a radionuclide, which when mixed with the humanized antibodies or humanized antibody fragments forms a conjugated system therewith. The above-noted buffers, excipients, and other component parts can be sold separately or together with the kit.

It will be recognized by one of skill in the art that the optimal quantity and spacing of individual dosages of a chimeric or humanized antibodies, antibody fragments or polypeptides of the embodiments described herein will be determined by the nature and extent of the condition being treated, the form, route and site of administration, and the particular animal being treated, and that such optimization can be determined by conventional techniques. It will also be appreciated by one of skill in the art that the optimal course of treatment, i.e., the number of doses of a chimeric or humanized antibodies, antibody fragments or polypeptides of the invention given per day for a defined number of days, can be ascertained by those skilled in the art using conventional course of treatment determination tests.

The subject chimeric or humanized antibodies, antibody fragments or polypeptides also may be administered in combination with other anti-cancer agents, e.g., other antibodies or drugs.

Additional embodiments include a recombinant antibody molecule having antigen binding regions derived from the heavy or light chain variable regions of an antibody which is capable of binding AF-20. Embodiments also include chimeric antibodies including variable regions obtained from a non-human antibody that binds AF-20 and human constant regions. It is preferred that the variable regions of the chimeric antibody are obtained from a murine antibody that binds AF-20 and human constant regions, and more preferably, from the murine monoclonal antibody (moAb) produced by hybridoma cell line ATCC designation HB 9686 and human constant regions.

Another embodiment includes a chimeric antibody comprising either a variable heavy chain sequence or a variable light chain sequence, or both. Preferably, the chimeric antibody is chNYR-1002.

Other embodiments encompass a humanized antibody or humanized antibody fragment (referred to collectively as “humoAb”) that binds AF-20, wherein the humanized antibody or humanized antibody fragment is derived from a non-human antibody that binds AF-20. Preferably, the humoAb is derived from a murine monoclonal antibody (moAb) that binds AF-20, and more preferably the humoAb is derived from the murine moAb produced by hybridoma cell line ATCC designation HB 9686. An especially preferred humanized antibody is huNYR-1002.

An additional embodiment includes a humanized antibody or humanized antibody fragment that binds AF-20 comprising Complementarity Determining Regions (CDRs) amino acid residues that are obtained from a non-human antibody that binds AF-20 and human Framework Regions (FRs) amino acid residues. Preferably, the CDRs are obtained from a murine moAb that binds AF-20 and human Framework Regions (FRs) amino acid residues, and more preferably, the CDRs are obtained from the murine moAb produced by hybridoma cell line ATCC designation HB 9686 and human Framework Regions (FRs) amino acid residues.

A particularly preferred humanized antibody or fragment includes one that binds AF-20 wherein the complementarity determining regions (CDR1, CDR2 and CDR3) of the light chain variable region and the complementarity determining regions (CDR1, CDR2 and CDR3) of the heavy chain variable region have the following amino acid sequences:

light chain:

| | | |
|---|---|---|
| CDR1 | (SEQ ID No. 44) | [RASQSIGTSIH]; |
| CDR2 | (SEQ ID No. 45) | [YASESIS]; |
| and | | |
| CDR3 | (SEQ ID No. 46) | [QQSSSWPFT]; |

heavy chain:

| | | |
|---|---|---|
| CDR1 | (SEQ ID No. 47) | [GYTFAGHYVH]; |
| CDR2 | (SEQ ID No. 48) | [WIFPGKVNTKYNEKFKG]; |
| and | | |
| CDR3 | (SEQ ID No. 49) | [VGYDYFYYFDY]. |

Embodiments described herein include humanized monoclonal antibodies or antibody fragments described above in which one or more amino acid residues in the variable regions or constant regions are replaced by other amino acid residues. Preferably, one or more amino acid residues in the CDRs or FRs are replaced by other amino acid residues. In addition, the embodiments include one or more additions, substitutions or deletions of amino acid residues made in the human Framework Regions (FRs).

Further embodiments described herein include humanized monoclonal antibodies or fragments thereof as described above, in which potential human helper T-cell epitopes identified in the variable regions have been removed by the substitution, addition or deletion of amino acid residues. Preferably, potential human helper T-cell epitopes identified in the CDRs or FRs have been removed by the substitution, addition or deletion of amino acid residues.

The humanized antibody or humanized antibody fragments (“humoAb”) described herein preferably have an antigen binding affinity for AF-20 that is at least 10% that of the antibody from which the humoAb was derived. A particularly preferred humoAb comprises either a humanized variable heavy chain sequence or a humanized variable light chain sequence, or both.

An additional embodiment includes a polypeptide sequence comprising one or more of the following polypeptides:

| | |
|---|---|
| SEQ ID No. 47 | [GYTFAGHYVH]; |
| SEQ ID No. 48 | [WIFPGKVNTKYNEKFKG]; |
| SEQ ID No. 49 | [VGYDYFYYFDY]; |
| SEQ ID No. 44 | [RASQSIGTSIH]; |
| SEQ ID No. 45 | [YASESIS]; |
| and/or | |
| SEQ ID No. 46 | [QQSSSWPFT]. |

Embodiments described herein also include a DNA encoding the antibody. polypeptide or antibody fragments described above, and any fragments, variants or derivatives thereof. Preferably, the DNA molecule encodes the amino acid sequence of a humanized antibody or fragment thereof, whereby the antibody or fragment specifically binds to AF-20, wherein the CDRs of the light chain variable region and the CDRs of the heavy chain variable region have the following amino acid sequences:

light chain:

| | | |
|---|---|---|
| CDR1 | (SEQ ID No. 44) | [RASQSIGTSIH]; |
| CDR2 | (SEQ ID No. 45) | [YASESIS]; |
| and | | |
| CDR3 | (SEQ ID No. 46) | [QQSSSWPFT]; |

heavy chain:

| | | |
|---|---|---|
| CDR1 | (SEQ ID No. 47) | [GYTFAGHYVH]; |
| CDR2 | (SEQ ID No. 48) | [WIFPGKVNTKYNEKFKG]; |
| and | | |
| CDR3 | (SEQ ID No. 49) | [VGYDYFYYFDY]. |

The embodiments also include DNA molecules that encode either the light or heavy chain of the above-described humoAb. A preferred DNA molecule encodes the amino acid sequence of a humanized antibody or fragment thereof, whereby the antibody or fragment specifically binds to AF-20, wherein the CDRs of the light chain variable region have the following amino acid sequences

| | | |
|---|---|---|
| CDR1 | (SEQ ID No. 44) | [RASQSIGTSIH]; |
| CDR2 | (SEQ ID No. 45) | [YASESIS]; |
| and | | |
| CDR3 | (SEQ ID No. 46) | [QQSSSWPFT]. |

Another preferred DNA molecule encodes the heavy chain of an antibody or fragment wherein the nucleotide sequences of the heavy chain CDRs are as follows:

| | | |
|---|---|---|
| CDR1 | (SEQ ID No. 47) | [GYTFAGHYVH]; |
| CDR2 | (SEQ ID No. 48) | [WIFPGKVNTKYNEKFKG]; |
| and | | |
| CDR3 | (SEQ ID No. 49) | [VGYDYFYYFDY]. |

Preferably, the DNA molecule is in the form of an expression vector. In this context, the embodiments further include a host transformed with the expression vector. In addition, the embodiments include a host cell comprising a recombinant expression system encoding the light and heavy chains of humanized antibody or humanized antibody fragments described above.

Other embodiments of the invention include nucleic acid sequence from which may be expressed a chimeric antibody described herein. In addition, the embodiments include a vector comprising the nucleic acid sequence. It is preferred that the vector is a bare nucleic acid segment, a carrier-associated nucleic acid segment, a nucleoprotein, a plasmid, a virus, a viroid, or a transposable element. Another preferred embodiment includes a hybridoma cell line that produces a chimeric antibodies described herein.

Additional embodiments include a nucleic acid sequence from which may be expressed a humanized antibody, humanized antibody fragment or polypeptide described above. In this context, the embodiments further include a vector comprising the nucleic acid sequence. Preferably, the vector is a bare nucleic acid segment, a carrier-associated nucleic acid segment, a nucleoprotein, a plasmid, a virus, a viroid, or a transposable element. Another preferred embodiment includes a hybridoma cell line that produces a humanized antibody, humanized antibody fragment or polypeptide described herein.

Certain embodiments include a composition for treating cancer comprising a therapeutically effective amount of any of the humanized or chimeric antibodies, humanized antibody fragments, or polypeptides described herein. Preferably, the humanized or chimeric antibody, humanized antibody fragment or polypeptide is, directly or indirectly, associated with or linked to an effector moiety having therapeutic activity. More preferably, the effector moiety is an anti-cancer drug, chemotherapeutic agent, cytotoxin, radionuclide, therapeutic enzyme, prodrug, cytokine, or anti-proliferative agent. Preferred radionuclide are ³²P, ⁴⁷Sc, ⁶⁷Cu, ⁹⁰Y, ¹⁰⁵Rh, ¹²⁵I, ¹³¹I, ^{117m}Sn, ¹⁵³Sm, ¹⁶⁶Dy, ¹⁷⁵Yb, ¹⁸⁶Re, ¹⁸⁸Re, ¹⁹⁴Os, ²¹¹At, ²¹²Bi, ²¹³Bi, ²²⁵Ac, or mixtures or combinations thereof.

Other embodiments encompass a method for in vivo treatment of a mammal having an AF-20-expressing cancer comprising administering to the mammal a therapeutically effective amount of the above-described composition. Preferably, the composition is administered post-operatively.

An additional embodiment includes a composition suitable for the in vivo or in vitro detection of cancer, the composition including a diagnostically effective amount of a humanized or chimeric antibody, humanized antibody fragment or polypeptide described herein. Preferably, the humanized or chimeric antibody, humanized antibody fragment or polypeptide is, directly or indirectly, associated with or linked to a detectable label. More preferably, the detectable label is a radionuclide, fluorescer, enzyme, enzyme substrate, enzyme cofactor, enzyme inhibitor, or ligand. Preferred radionuclides include ³H, ¹¹C, ¹⁴C, ¹⁸F, ⁶⁴Cu, ⁷⁶Br, ⁸⁶Y, ^{99m}Tc, ¹¹¹In, ¹²³I, ¹⁷⁷Lu, and mixtures and combinations thereof.

Further embodiments include a method for in vitro immunodetection of AF-20-expressing cancer cells comprising contacting the cancer cells with the composition described immediately above. In this embodiment, it is preferred that the humanized or chimeric antibodies, humanized antibody fragment or polypeptides of the composition are bound to a solid support.

Another preferred method includes a method of in vivo immunodetection of AF-20-expressing cancer cells in a mammal comprising administering to the mammal a diagnostically effective amount of the composition described above that is suitable for detection of cancer. Preferably, the immunodetection procedure is in vivo tumor imaging.

An additional embodiment includes a method of in vivo treatment of cancer comprising (i) intravenously administering a radionuclide-labeled antibody, antibody fragment or polypeptide, (ii) thereafter detecting tumor cells using a radionuclide activity probe, and (iii) thereafter removing the detected tumor cells by surgical excision. In this method, it is preferred that the antibody or polypeptide is a humanized or chimeric antibody, humanized antibody fragment or polypeptide described above. Preferably, the radionuclide is ³H, ¹¹C, ¹⁴C, ¹⁸F, ⁶⁴Cu, ⁷⁶Br, ⁸⁶Y, ^{99m}Tc, ¹¹¹In, ¹²³I, ¹⁷⁷Lu, or mixtures and combinations thereof.

Embodiments of the invention further include a method of using a humanized or chimeric antibody, humanized antibody fragment or polypeptide described above, or a DNA molecule described above to generate polypeptides or variants or derivatives of the antibodies, fragments or polypeptides that bind AF-20. Preferably, the method of generating the polypeptides, variants or derivatives is phage or yeast display technology.

Embodiments of the invention now will be explained with reference to the following non-limiting examples.

### EXAMPLES

### Example 1

### Sequencing of Murine Antibody Genes

The murine hybridoma AD20D4 was revived and cultured as directed in Dulbecco's Modification of Eagle's Medium with Glutamax I (Invitrogen Corp. Cat No. 61965-026, Lot No. 3070663) supplemented with 20% fetal bovine serum of North American origin (Invotrogen Corp. Cat. No. 16000-044, Batch No. 1137907) and 1 mM Sodium pyruvate Cat. No. 11360-039, Lot No. 3069371).

Total RNA was prepared from 10⁷ hybridoma cells, taking care to avoid contamination with RNAses. Special RNAse free reagents were used including nuclease-free water. The cells were spun down to collect in a MSE 2000R refrigerated bench centrifuge at 1500 rpm for 5 minutes at 4° C. then washed three times in ice-cold PBS. The cells were then resuspended in 6 mL in ice-cold RNA lysis buffer (0.14M NaCl, 1.5 mM MgCl₂, 10 mM Tris pH 8.6, 0.5% NP-40) to which 5 μL RNAseOUT had been added and vortexed for ten seconds. This solution was overlayed onto an equal volume of 24% (w/v) sucrose and 1% NP-40 and stored on ice for five minutes. The solution was then centrifuged at 4000 rpm for 30 minutes at 4° C. in a refrigerated bench centrifuge. The upper cytoplasmic phase was then removed to an equal volume of 2× PK buffer (0.3 M NaCl, 0.025M EDTA, 0.2M Tris pH 7.5, 2% SDS) and proteinase K (Life Technologies Cat. No. 25530-049) was added to a final concentration of 200 μg/mL. The solution was incubated at 37° C. for 30 minutes.

The solution then was extracted with an equal volume of phenol/chloroform (1:1 (w/v)). To the aqueous phase were added 2.5 volumes of 100% ethanol and the solution stored at −20° C. overnight. The RNA was collected by centrifugation (400 rpm for 30 minutes) then dried in a vacuum dessicator. The RNA was dissolved in H₂O (Promega Cat. No. P119C) and the concentration measures by spectrometry assuming A260 1=40 μg/mL. 1-2 μg were run on a 1.2% agarose gel in TAE to confirm the quality of RNA: good quality RNA will show sharp ribosomal bands with no evidence of degradation.

V_{H} and V_{K} cDNAs were prepared using reverse transcriptase with mouse IgG constant region and mouse K constant region primers, taking care to avoid contamination with RNAses. First strand variable region cDNA was prepared by mixing in a microcentrifuge tube 5 μg RNA, 10 μL 5× reverse transcriptase buffer (Promega Cat. No. M351A), 1 μL primer (25 pmol/μL in H₂O (Promega Cat. No. P119C) using MuIgGVH3′ (Oligo No. 152) for heavy chain; MuIgKVL3′ (Oligo No. 160) for light chain), 2 μL 10 mM dNTPs solution (10 mM each of dATP, dCTP, dGTP, dTTP from 100 mM stock solutions (Life Technologies Cat. No. 10297018)), 2 μL RNAseOUT (Life Technologies Cat. No. 10777019) and H₂O (Promega Cat. No. P119C) to 50 μL. The solution was heated to 70° C. for ten minutes then cooled slowly to 37° C. 100 units M-MLV reverse transcriptase (Promega Cat. No. M530A) was added and the solution incubated at 37° C. for one hour, heated to 70° C. for 15 minutes then stored at −20° C. until required.

The amplification and cloning of the variable region genes then was carried out.

In a microcentrifuge tube were mixed 5 μL 1^{st} strand cDNA, 5 μL 10× Taq polymerase buffer (Life Technologies Cat. No. 402028), 1 μL 3′ primer (25 pmol/μL in H₂O using MuLgVH3′ (oligo No. 152) for heavy chain; MuIgKVL3′ (Oligo No. 160) for light chain)), 1 μL 5′ leader primer mix (25 pmol/μL of each primer in the mix), 1 μL 10 mM dNTPs solution, 0.5 μL Taq polymerase buffer (Life Technologies Cat. No. 10342-020) and H₂O to 50 μL. All reagents except the Taq enzyme were mixed in a 0.5 mL thin wall PCR tube and heated to 94° C. on the PCR block. The Taq enzyme was added then the samples were cycled: 94° C./2 min, 40 cycles of 94° C./30 sec, 50° C./30 sec, 72° C./2 min, finishing with 72° C. for five minutes. 5 μL of each reaction was run on an agarose gel to check that the PCRs have given products of the expected size (approximately 350 bp). The remainder of the product was loaded onto a 1.5% low-melting point agarose gel and the DNA bands excised and purified. The gel purified V region DNA was ligated into 1 μL pGem T-easy cloning vector (Promega Cat No. A1360) by mixing with 2 μL 10× T4 DNA ligase buffer (Promega Cat. No. C126B) and 1 μL T4 DNA ligase (promega Cat. No. M180A) then incubated at 15 to 20° C. for two hours to overnight. The vector was transformed into competent E. Coli TG1 and plated onto LB+IPTG+XGAL+ampicillin plates. White colonies to 3 mLs of LB+ampicillin were picked in universal containers and grown at 37° C. After two to four hours inserts were checked for by test PCR. 50 μL of culture was removed to a microfuge tube and heated to 95° C. for five minutes. It was then spun in microfuge for five minutes and the supernatant removed to a fresh tube. In 10 μL of supernatant was mixed 5 μL 10× Taq polymerase buffer, 1 μL M13 forward primer, 1 μL M13 reverse primer, 1 μL 10 mM dNTPs, 0.5 μL Taq polymerase and H₂O to 50 μL.

All reagents except the Taq enzyme were mixed in a 0.5 mL thin wall PCR tube and heated to 94° C. on the PCR block. The Taq enzyme was added then the samples were cycled: 94° C./2 min, 40 cycles of 94° C./30 sec, 50° C./30 sec, 72° C./2 min, finishing with 72° C. for five minutes. 10 μL of each reaction was run on an agarose gel to check for inserts (band at 500 bp). The cultures were grown overnight and the DNA was prepared for DNA sequencing.

The DNA sequence of selected clones (V_{H} and V_{K} clones screened for inserts of the expected size by PCR) was determined by automated DNA sequencing. Plasmid DNA was prepared from the screened bacterial stock, Five mL cultures were set up in Luria broth (LB) (NaCl 10 g, Tryptone 10 g, yeast extract 5 g per litre H₂O) with 50 μg/mL (or as required) ampicillin stock (Sigma Cat. No. A-0166) (50 mg/mL in H₂O) in universal containers. The cultures were grown shaking overnight or for at least five hours. The culture was spun down in microfuge and the DNA purified using Wizard Plus SV mini-prep kits (Promega Cat. No. A1460), following the manufacturer's instructions. The purified DNA was then resuspended in 100 μL H₂O and sequenced using automated DNA sequencing equipment.

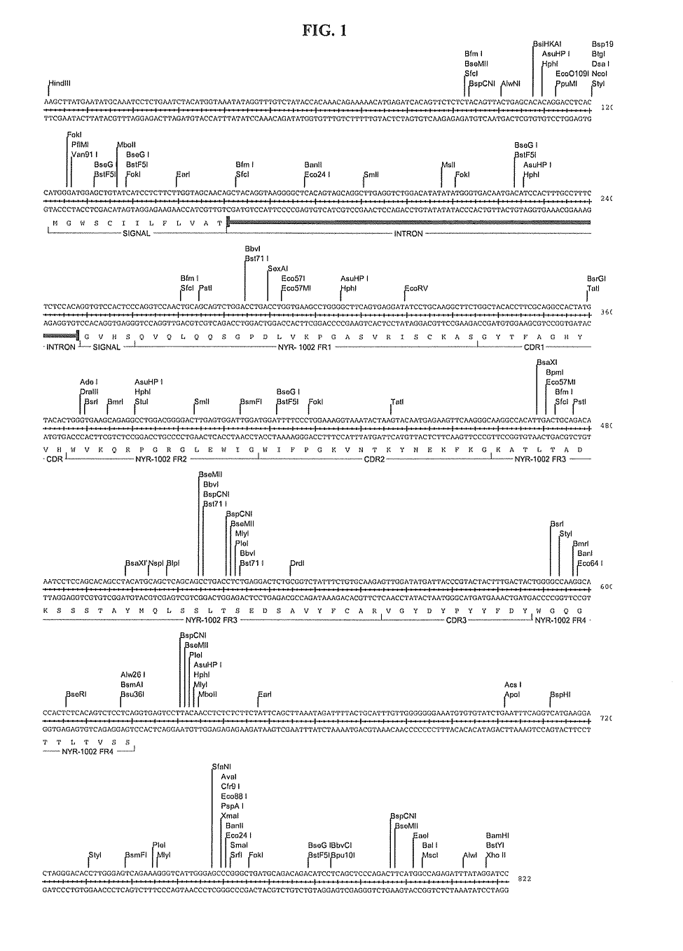

The DNA and amino acid sequence for the NYR-1002 heavy chain V region is shown in FIG. 1. No productive heavy chain genes were isolated from the first batch of cells. From a second batch, fifteen independent clones gave identical complete heavy chain sequences. The location of the CDRs was determined with reference to other antibody sequences (Kabat E A et al., 1991). The CDRs are given by SEQ ID No. 47 (CDR1), 48 (CDR2) and 49 (CDR3). The NYR-1002 V_{H} amino acid sequence was compared to the consensus sequence for Mouse Heavy Chains Subgroup IIB and was assigned to that Subgroup.

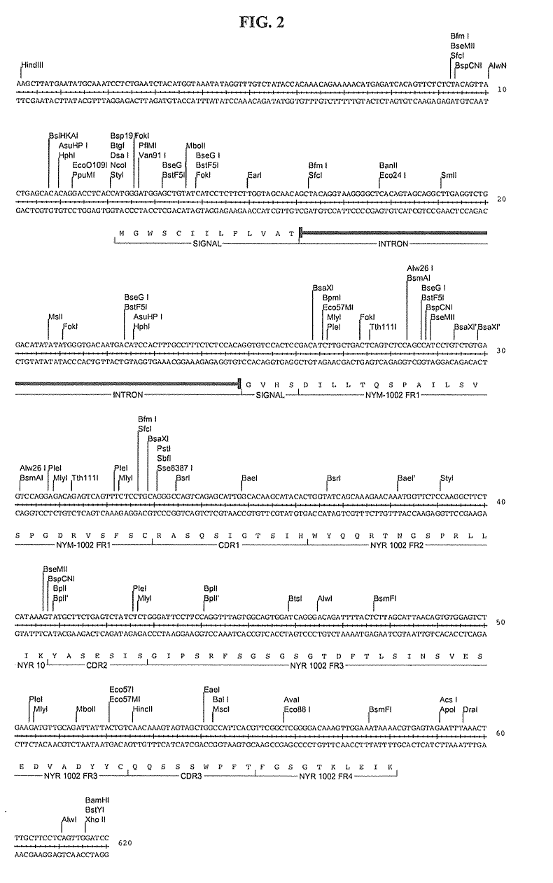

The DNA and amino acid sequence for the NYR-1002 light chain V region is shown in FIG. 2. Five independent clones from each batch of cells gave identical sequences. The location of the CDRs was determined with reference to other antibody sequences (Kabat E A et al., 1991). The CDRs are given by SEQ ID No. 44 (CDR1), 45 (CD ) and 46 (CDR3). The NYR-1002 V_{H} amino acid sequence was compared to the consensus sequence for Mouse Kappa Chains Subgroup V and was assigned to that Subgroup.

### Example 2

### Construction of Chimeric Antibody Genes and Chimeric Antibody

A chimeric antibody was constructed by linking the murine variable regions identified in Example 1 above to human constant regions. The murine variable regions were appended by the method of overlapping PCR recombination as described by Orlandi et al. (1989). Also See Daughterty B L et al. (1991). The cloned murine V_{H} and V_{K} genes were amplified. The vectors VH-PCR1 and VK-PCR1 (Riechmann et al. 1988) were used as templates to introduce 5′ flanking sequence, the leader intron and the murine immunoglobulin promoter and 3′ flanking sequence including the splice site and intron sequences. The V_{H} and V_{K} expression cassettes produced were cloned into pUC19 and the entire DNA sequence confirmed to be correct by sequencing. The PCR amplification was conducted as follows: a set of mutagenic oligonucleotides, all at 25 pmol/μL, were synthesized. This set encompassed the site to be mutated such that the DNA sequence is amplified as a set of fragments. Depending on the number of sites to be mutated, adjacent oligonucleotides were designed.

PCR amplifications were set up for each primer pair: 1 μL template DNA was mixed with 5 μL 10× Pfu polymerase buffer (Stratagene Cat. No.600153-82 or Promega Cat. No. M776A), 1 μL (25 pmol/μL) forward primer, 1 μL (25 pmol/μL) reverse primer, 2 μL 10 mM dNTPs, 0.5 μL (1 unit) Pfu DNA polymerase (Stratagene Cat. No. 600252-51 or Promega Cat. No. M774A) and H₂O to 50 μL. These 5′ and 3′ primers included a terminal 18 bp of random sequence. All reagents except the Pfu enzyme were mixed in a 0.5 mL thin wall PCR tube and heated to 94° C. on the PCR block. The Pfu enzyme was added then the samples were cycled: 94° C./2 min, 15-20 cycles of 94° C./30 sec, 50° C./30 sec, 72° C./1 min (depending on the length of extension required), finishing with 72° C. for five minutes. The annealing temperature was adjusted either higher or lower than 50° C. depending on the Tₘ of the oligos. Five μL of each reaction was run on an agarose gel to check for products of the expected size. If not, the annealing temperature was lowered by 5° C., the number of cycles of PCR was increased, and/or the MgCl₂ concentration was increased to 5 mM. If this round of PCR yielded multiple bands, it was necessary to gel-purify the band of the correct size.

The products were joined in a second PCR using second round 5′ and 3′ primers comprising the terminal 18 bp added in the first round of PCR. The template for the second joining PCR is the fragments produced in the first round with the quantities adjusted to add approximately equal amounts. The products of the first round of PCR were mixed with 5 μL 10× Pfu polymerase buffer (Stratagene Cat. No. 600153-82 or Promega Cat. No. M776A), 2 μL (50 pmol/μL) 5′ 2^{nd} round primer, 2 μL (50 pmol/μL) 3′ 2nd round primer, 2 μL 10 mM dNTPs, 0.5 μL (1 unit) Pfu DNA polymerase (Stratagene Cat. No. 600252-51 or Promega Cat. No. M774A) and H₂O to 50 μL. All reagents except the Pfu enzyme were mixed in a 0.5 mL thin wall PCR tube and heated to 94° C. on the PCR block. The Pfu enzyme was added then the samples were cycled: 94° C./2 min, 15 cycles of 94° C./30 sec, 50° C./30 sec, 72° C./1 min (depending on the length of extension required), finishing with 72° C. for five minutes. Five μL of each reaction was run on an agarose gel to check for products of the expected size (approximately 820 bp for V_{H} expression cassettes and 650 bp for V_{K} expression cassettes. If not, the 2^{nd} round PCR was repeated lowering annealing temperature by 5° C. and/or increasing the number of cycles of PCR. The PCR product was extracted and precipitated using phenol/chloroform and ethanol or Qiagen MiniElute PCR Purification kit (Cat. No. 28004). The resulting product was digested with the required enzymes (HindIII and BamHI for expression cassettes) and loaded onto a 1.5% low-melting point agarose gel. The DNA bands of the correct size were excised and purified. The DNA was sequenced to confirm that it was correct and that no spurious mutations had been introduced.

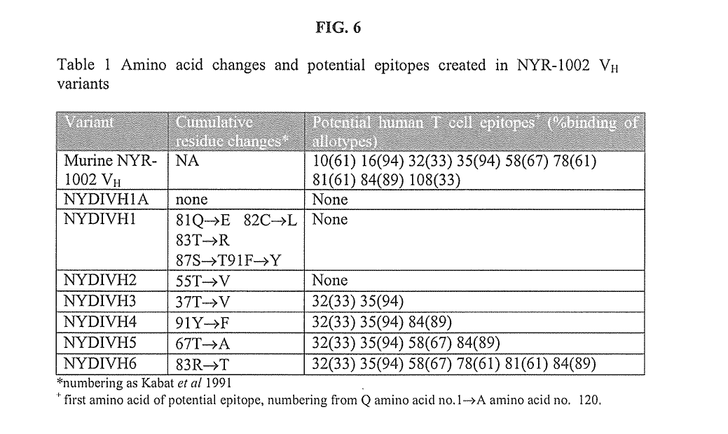

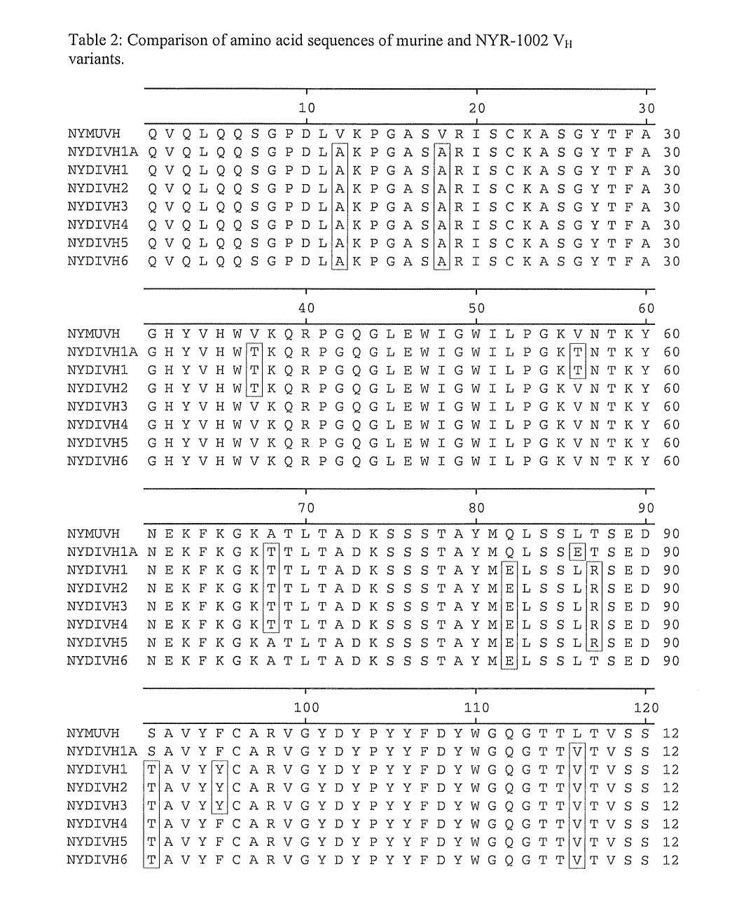

The heavy and light chain V-region genes were transferred to the expression vectors pSVgpt and pSVhyg, which include human IgG1 or K constant regions respectively and markers for selection in mammalian cells. The Antibody Heavy Chain Expression Vector is illustrated in FIG. 6. Sites in brackets in the figure have been removed. The heavy chain expression vector pSVgptHuIgG1 is based on pSV₂gpt (Mulligan and Berg, Science (1980; 209:1422-1427)). It includes the ampicillin resistance gene for selection in bacterial cells, the gpt gene for selection in mammalian cells, the murine heavy chain immunoglobulin enhancer region, genomic sequence encoding the Human IgG1 constant region gene and SV40 poly A sequences. The heavy chain variable region for expression is inserted as a HindIII to BamHI fragment. This expression cassette includes the murine heavy chain promoter, the signal peptide coding sequence and the signal sequence intron, the V_{H} gene, the V-C splice donor sequence and intron sequences.

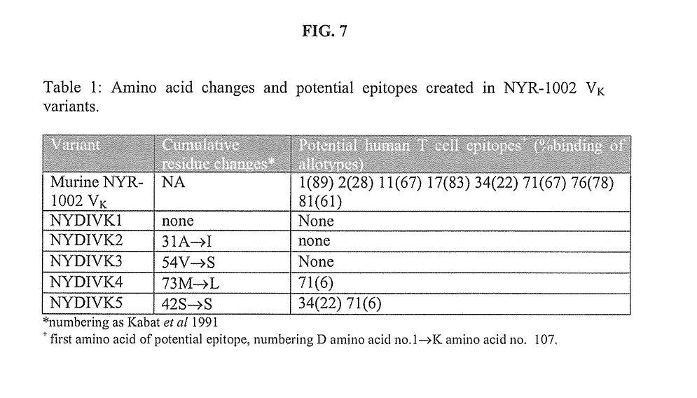

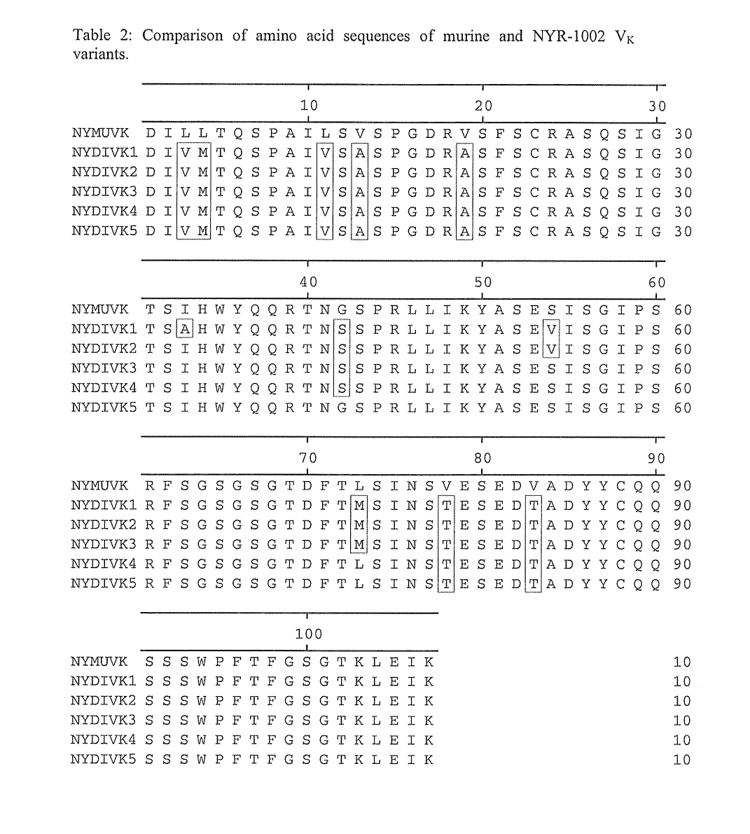

The Antibody Light Chain Expression Vector is illustrated in FIG. 7. Sites in brackets in the figure have been removed. There are 3EcoRI sites internal to HuC_{κ}. The light chain expression vector pSVgptHuC_{κ} is based on the vector pSVhyg. It includes the ampicillin resistance gene for selection in bacterial cells, the hyg gene for selection in mammalian cells, the murine heavy chain immunoglobulin enhancer region, genomic sequence encoding the Human kappa constant region gene and including the kappa enhancer and SV40 poly A sequences. The light chain variable region for expression is inserted as a HindIII to BamHI fragment. This expression cassette includes the murine heavy chain promoter, the signal peptide coding sequence and the signal sequence intron, the V_{κ} gene, the V-C splice donor sequence and intron sequences. The DNA sequence was confirmed to be correct for the V_{H} and V_{κ} in the chimeric expression vectors.

The heavy and light chain expression vectors were co-transfected into NS0 cells (European Collection of Animal Cell Cultures, Porton, UK, ECACC No. 85110503) by electroporation. Pvul digests of about 3 and 6 μg respectively of the pSVgptHuIgG1 and pSVgptHuC plasmids. The digested DNA was ethanol precipitated and dissolved in 20 μL dH₂O. The cells were resuspended from a semi-confluent 75 cm³ flask and collected by centrifugation at 1000 rpm for five minutes. The supernatant was discarded. The cells were resuspended in 0.5 mL Dulbecco's Modified Eagle's Medium (DMEM) and transferred to a Gene Pulser cuvette (Bio-Rad). The DNA was mixed with the cells by gentle pipetting and left on ice for five minutes. The cuvette was inserted between the electrodes of the Bio-Rad Gene Pulser and a single pulse of 170 V, 960 μF applied. The cuvette was returned to ice for 20 minutes. The cell suspension then was transferred to a 75 cm³ flask containing 20 mL DMEM and allowed to recover for 1-2 days. The cells then were harvested and resuspended in 80 mL selective DMEM and aliquot 200 μL to each well of the 96-well plates.

Approximately 10 days later, 20 μL of medium from each well was assayed for the presence of human antibodies and wells were chosen for expansion based on the level of antibody production and the number of cells in the well. Cells from chosen wells were resuspended by rubbing the tip of a Gilson P200 pipette (with yellow tip) across the surface and the medium transferred to a well of a 24-well tissue culture plate containing 1.5 mL of fresh selective DMEM. Colonies expressing the gpt gene were selected in DMEM supplemented with 10% fetal bovine serum, 0.8 μg/mL mycophenolic acid and 250 μg/mL xanthine. Transfected clones were screened for production of human antibody by ELISA for human IgG. Cell lines secreting antibody were expanded and the highest producers selected and frozen down in liquid nitrogen. The chimeric antibody was purified using Prosep®-A (Millipore Corp.). The concentration was determined by ELISA for human IgG1_{κ}.

### Example 3

### Identification of Human Helper T Cell Epitopes Contained Within the Variable Regions of Mouse NYR-1002

The amino acid sequences determined in Example 1 were analyzed to produce human T cell epitope maps of the variable region using Peptide Threading software (Biovation). FIG. 5 shows the results of this analysis. The analysis showed a total of 17 potential human T cell epitopes in NYR-1002, 9 in V_{H} and 8 in V_{K}. None of the potential T cell epitopes occurred entirely coincident with a CDR.

### Example 4

### Design of Modified Antibody Sequences

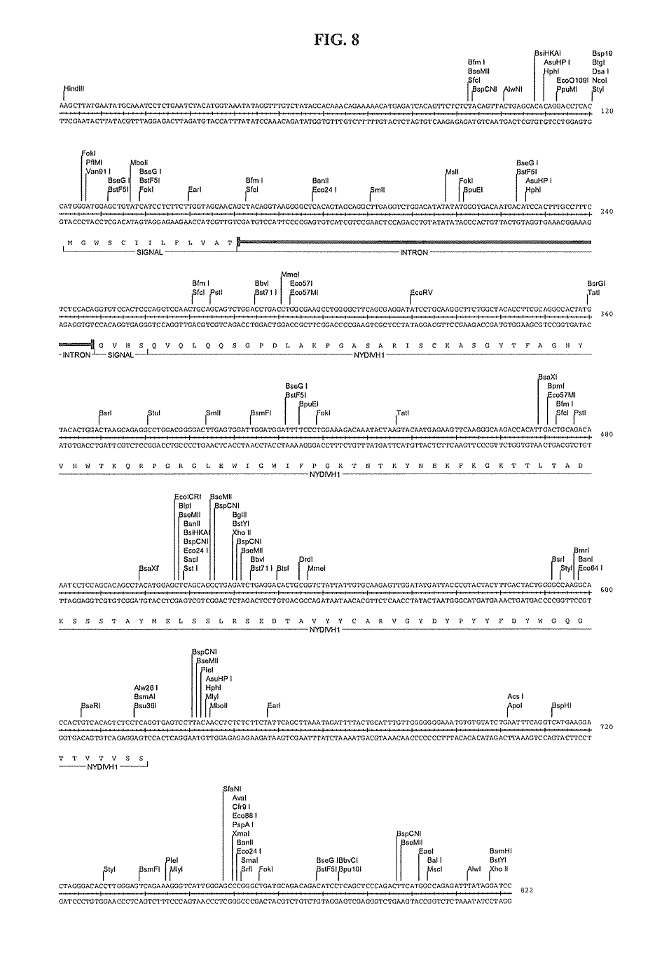

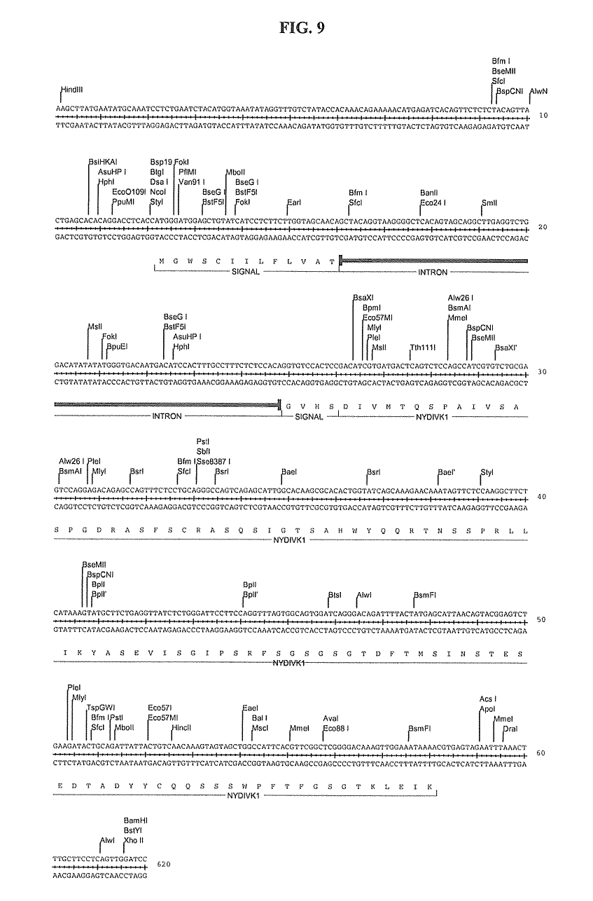

Primary V_{H} and V_{K} variant sequences (NYDIVH1, NYDIVK1) were designed by substituting amino acid residues in the variable regions of the murine AF-20 antibody in order to remove potential human T cell epitopes but retaining, where required, critical amino acids: See FIGS. 6, 7, 8 and 9. The DNA and amino acid sequences for the primary V_{H} region is shown in FIG. 8 and for the primary V_{K} region in FIG. 9.

As the generation of the primary V_{H} and V_{K} variant sequences required a small number of amino acid substitutions that might have affected the binding of the final polypeptide, six other variant V_{H}s (designated NYDIVH1A, NYDIVH2, NYDIVH3, NYDIVH4, NYDIVH5 and NYDIVH6) and 4 other V_{K}s (designated NYDIV , NYDIVK3, NYDIVK4, and NYDIVK5) were designed. The comparative amino acid sequences of the murine and DeImmunised V regions are shown in FIG. 6 for V_{H} and FIG. 7 for V_{K}. The altered amino acid sequence for the variant V_{H} and V_{K}s re-introduced some potential T cell epitopes (Table 1 of FIGS. 6 and 7, respectively).

### Example 5

### Construction of Modified Antibody Sequences

The modified variable regions were constructed by the method of overlapping PCR recombination as described by Orlandi et al (1989) and as detailed in Example 2 above. The cloned murine V_{H} and V_{K} genes were used as templates for mutagenesis of the framework regions to the required sequences. Sets of mutagenic primer pairs were synthesized encompassing the regions to be altered. The vectors VH-PCR1 and VK-PCR1 (Riechmann et al., 1988) were used as templates to introduce 5′ flanking sequence including the leader signal peptide sequence, the leader intron and the murine immunoglobulin promoter, and 3′ flanking sequence including the splice site and intron sequences. The modified V_{H} and V_{K} expression cassettes produced were cloned into pUC19 and the entire DNA sequence was confirmed to be correct for each modified V_{H} and V_{K} sequence.

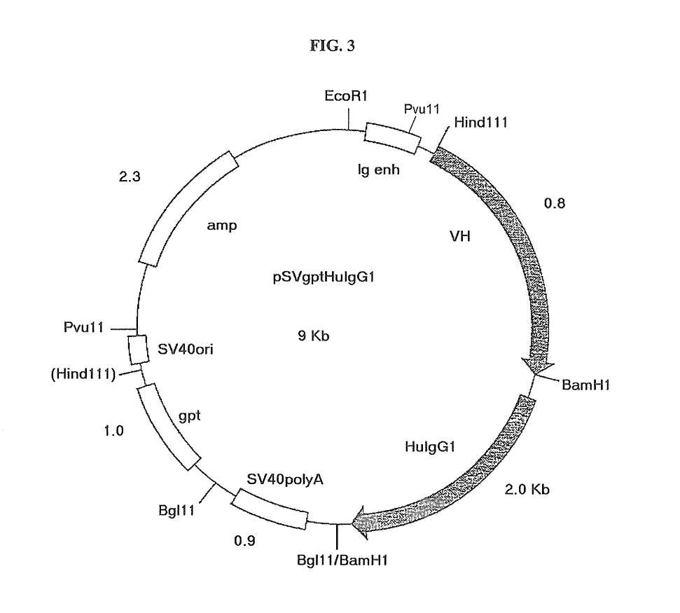

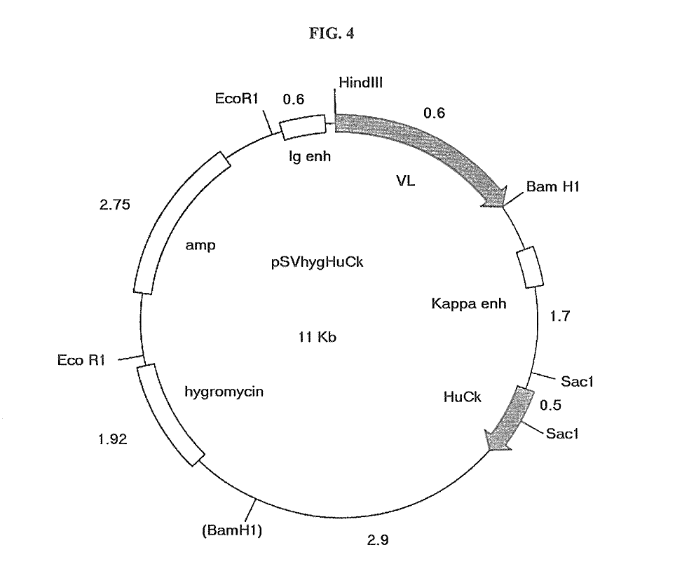

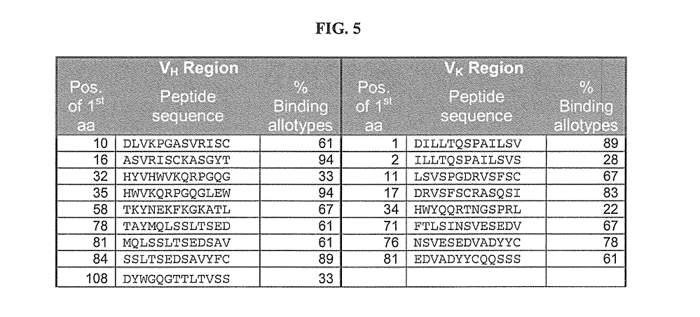

The modified heavy and light chain V-region genes were excised from pUC19 as HindIII to BamHI expression cassettes. These were transferred to the expression vectors pSVgpt and pSVhyg (FIG. 3 and 4 respectively), which include human IgG1 or K constant regions respectively and markers for selection in mammalian cells. The DNA sequence was confirmed to be correct for the modified V_{H} and V_{K} sequences in the expression vectors.

The host cell line for antibody expression was NS0, a non-immunoglobulin producing mouse myeloma, obtained from the European Collection of Animal Cell Cultures, Porton UK (ECACC No 85110503). The heavy and light chain expression vectors were co-transfected into NS0 cells by electroporation (see EXAMPLE 2 above). Colonies expressing the gpt gene were selected in Dulbecco's Modified Eagle's Medium (DMEM) supplemented with 10% fetal bovine serum, 0.8 μg/ml mycophenolic acid and 250 μg/ml xanthine. Transfected cell clones were screened for production of human antibody by ELISA for human IgG. Cell lines secreting antibody were expanded and the highest producers selected and frozen down in liquid nitrogen. The modified antibodies were purified using Prosep®-A (Bioprocessing Ltd). The concentration was determined by ELISA for human IgG1κ. The antibodies were also analysed by SDS-PAGE.

### Example 6

### Expression of Modified Antibodies

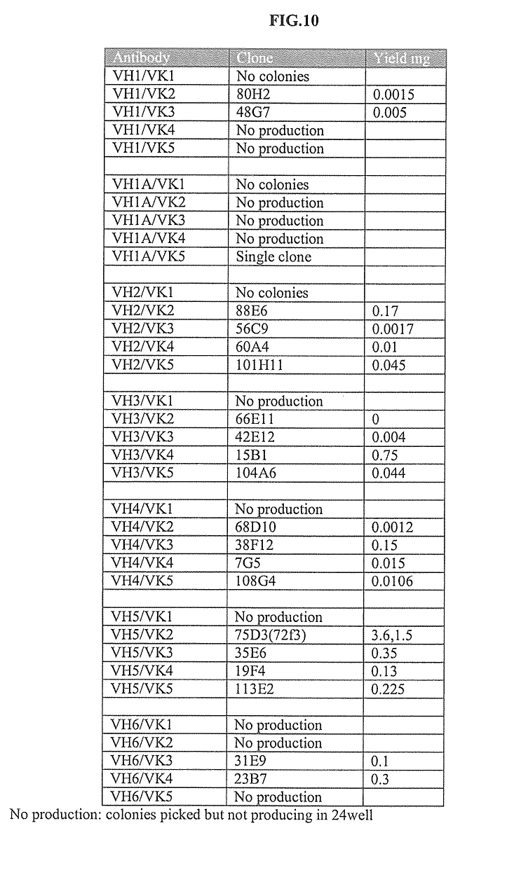

The transfected cell clones were tested for antibody expression. Combinations of most heavy and light chains were poor producers (See FIG. 10). NYDIVH5 when combined with NYDIVK2 was the best producer, producing 3.6 mg of purified antibody. For comparison, the chimeric antibody yielded 0.66 mg of purified protein. NYDIVH1A when combined with any light chain failed to produce any antibody.

Antibodies comprising NYDIVH2/NYDIVK2, NYDIVH2/NYDIVK3 performed the best when assayed, with NYDIVH4/NYDIVK5, NYDIVH4/NYDIVK3 being slightly weaker. NYDIVH2/NYDIVK2 was selected as the lead humanized antibody.

### Example 7

### Human T Cell Assay of Modified Antibody and of Chimeric Antibody

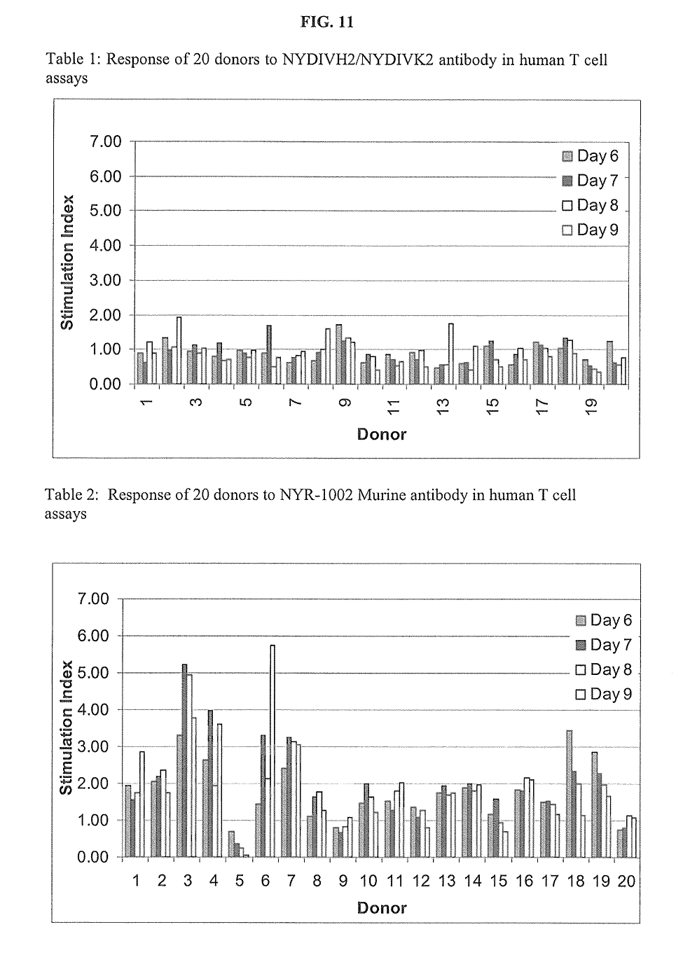

The humanized antibody NYDIVH2/NYDIVK2 (huNYR-1002) together with the chimeric antibody (chNYR-1002) were tested in human T cell proliferation assays. Buffy coats from healthy donors were used to isolate peripheral blood mononuclear cells (PBMC) which contain antigen presenting cells (APCs) and T cells. MHC class II allotypes of these donors were determined and 20 donors were selected resulting in >80% HLA DRB1 allotypic coverage for the Caucasian population. As shown in Table 1 of FIG. 11, none of 20 donors gave a significant response (Stimulation index, SI>2) to the humanized antibody, huNYR-1002, in the human T cell proliferation assay. In comparison 12 donors responded to murine IgG, chNYR-1002, with SI's>2, although 5 of these donors produced borderline responses with an SI of 2-2.5 (Table 2 of FIG. 11). The clear differential response to the humanized antibody validates the effect of the amino acid substitutions in the murine variable regions in reducing T cell immunogenicity.

### Example 8

### Determination of the Cytotoxic Efficacy of the Modified Antibody Conjugated to Cytotoxic Agents

Antibody (huNYR-1002) was conjugated to various known cytotoxic molecules and tested for cytotoxicity effects in cultures of cancer cells. Cytotoxic compounds (such as methotrexate and doxorubicin) were conjugated to the antibody by maleimide activation using Sulfosuccinimidyl 4-[N-maleimidomethyl]-cyclohexane-1-carboxylate and size exclusion chromatographic purification. Substitution ratios of 2-10 moles of compound per mole of antibody were used.
variations in conjugation methodology are contemplated in the embodiments disclosed herein, and are capable of being achieved by those skilled in the art, using the guidelines provided herein;
variations in choice of conjugated cytotoxic molecule are contemplated in the embodiments disclosed herein, and are well within the purview of one skilled in the art, using the guidelines provided herein.

96 well plates were seeded with cells (eg CCL-185 cells) at 10³-10⁵ cells/well. Perimeter wells were free of cells. Blank well controls were filled with compound alone, media alone, and compound plus media. Controls also included cells plus media alone, and cells alone. Compounds were tested in the 0.01 to 0.25 μg/mL concentration range. Plates were incubated for 4 days and then were tested by various viability assays according to manufacturer's directions. (eg. Celltiter 96 Aqueous One Solution (MTS)). Following addition of stain or reagent to the cell wells, plates were incubated for 1-4 hours at 37° C., mixed for several seconds, and then read for absorbance on a plate reader.

Results:

The newly prepared compounds (conjugates of antibody to cytotoxic molecule) were found to be more toxic (per mole of cytotoxic compound) than the unconjugated cytotoxic compounds. The increased cytotoxicity of the compound conjugated to antibody ranged from 10-10⁴× potency (for an equal cytotoxic effect, the unconjugated compound required 10-10⁴× more molecules of compound, as compared to the compound conjugated to antibody). Maximal effect was seen after 4 days of culture. These results indicated that 1) the antibody conjugates provided equal cytotoxicity at lower concentrations of cytotoxic compound, implying less toxicity to non-cancerous cells and tissues where the antibody binds less; 2) the antibody conjugates were more cytotoxic at lower concentrations, implying greater efficacy at killing the cancer cells; and 3) the cytotoxic compounds are sequestered and conjugated to the monoclonal antibody, implying less toxicity to non-cancerous cells and tissues where the antibody binds less.

What is claimed is:

1. The chimeric antibody chNYR-1002.

2. A humanized antibody or humanized antibody fragment that binds AF-20 wherein said humanized antibody or humanized antibody fragment is derived from a non-human antibody that binds AF-20.

3. The humanized antibody or humanized antibody fragment (humoAb) of claim 2, wherein the humoAb is derived from a murine monoclonal antibody (moAb) that binds AF-20.

4. The humanized antibody or humanized antibody fragment of claim 3, wherein the humoAb is derived from the murine moAb produced by hybridoma cell line ATCC designation HB 9686.

5. The humanized antibody huNYR-1002.

6. A humanized antibody or humanized antibody fragment that binds AF-20 comprising Complementarity Determining Regions (CDRs) amino acid residues that are obtained from a non-human antibody that binds AF-20 and human Framework Regions (FRs) amino acid residues.

7. The humanized antibody or humanized antibody fragment of claim 6, wherein the Complementarity Determining Regions (CDRs) amino acid residues are obtained from a murine moAb that binds AF-20.

8. The humanized antibody or humanized antibody fragment as claimed in claim 7, wherein the murine moAb that binds AF-20 is produced by hybridoma cell line ATCC designation HB 9686 and human Framework Regions (FRs) amino acid residues.

9. A humanized antibody or fragment thereof that binds AF-20 wherein the complementarity determining regions (CDR1, CDR2 and CDR3) of the light chain variable region and the complementarity determining regions (CDR1, CDR2 and CDR3) of the heavy chain variable region are comprised of the following amino acid sequences:
light chain:

| | | |
|---|---|---|
| CDR1 | (SEQ ID No. 44) | [RASQSIGTSIH] |
| CDR2 | (SEQ ID No. 45) | [YASESIS] |
| CDR3 | (SEQ ID No. 46) | [QQSSSWPFT] |

heavy chain:

| | | |
|---|---|---|
| CDR1 | (SEQ ID No. 47) | [GYTFAGHYVH] |
| CDR2 | (SEQ ID No. 48) | [WIFPGKVNTKYNEKFKG] |
| CDR3 | (SEQ ID No. 49) | [VGYDYFYYFDY]. |

10. The humanized antibody or humanized antibody fragment of claim 9 wherein one or more additions, substitutions or deletions of amino acid residues have been made in the human Framework Regions (FRs).

11. The humanized antibody or fragment of claims 9, wherein potential human helper T-cell epitopes identified in the variable regions have been removed by the substitution, addition or deletion of amino acid residues.

12. The humanized antibody or humanized antibody fragment of claim 2, wherein the antibody has an antigen binding affinity for AF-20 which is at least 10% that of the antibody from which the humanized antibody or humanized antibody fragment was derived.

13. A polypeptide sequence comprising a sequence selected from at least one of the group consisting of:

| | | |
|---|---|---|
| a) | SEQ ID No. 47 | [GYTFAGHYVH]; |
| b) | SEQ ID No. 48 | [WIFPGKVNTKYNEKFKG]; |
| c) | SEQ ID No. 49 | [VGYDYFYYFDY]; |
| d) | SEQ ID No. 44 | [RASQSIGTSIH]; |
| e) | SEQ ID No. 45 | [YASESIS]; |
| and | | |
| f) | SEQ ID No. 46 | [QQSSSWPFT]. |

14. A DNA encoding the polypeptide sequence of claims 13.

15. A DNA molecule encoding the amino acid sequence of the humanized antibody or fragment of claim 9.

16. A DNA molecule encoding the light chain of an antibody or fragment as claimed in claim 2.

17. The DNA molecule as claimed in claim 16, wherein the nucleotide sequences of the light chain CDRs are as follows:

| | | |
|---|---|---|
| CDR1 | (SEQ ID No. 44) | [RASQSIGTSIH]; |
| CDR2 | (SEQ ID No. 45) | [YASESIS]; |
| and | | |
| CDR3 | (SEQ ID No. 46) | [QQSSSWPFT]. |

18. A DNA molecule encoding the heavy chain of an antibody or fragment as claimed in claim 2.

19. A DNA molecule as claimed in claim 18, wherein the nucleotide sequences of the heavy chain CDRs are as follows:

| | | |
|---|---|---|
| CDR1 | (SEQ ID No. 47) | [GYTFAGHYVH]; |
| CDR2 | (SEQ ID No. 48) | [WIFPGKVNTKYNEKFKG]; |
| and | | |
| CDR3 | (SEQ ID No. 49) | [VGYDYFYYFDY]. |

20. The DNA molecule as claimed in claim 15 in the form of an expression vector.

21. A host transformed with the expression vector of claim 20.

22. A host cell comprising a recombinant expression system encoding the light and heavy chains of an antibody or antibody fragment of claim 9.

23. A hybridoma cell line that produces the humanized antibody or antibody fragment of claim 2.

24. A composition for treating cancer comprising a therapeutically effective amount of a humanized antibody or humanized antibody fragment according to claim 2.

25. The composition of claim 24, wherein the humanized antibody or humanized antibody fragment is, directly or indirectly, associated with or linked to an effector moiety having therapeutic activity.

26. The composition of claim 25, wherein the effector moiety is selected from the group consisting of an anti-cancer drug, chemotherapeutic agent, cytotoxin, radionuclide, therapeutic enzyme, prodrug, cytokine, an anti-proliferative agent, and mixtures thereof.

27. The composition of claim 26, wherein the radionuclide is selected from the group consisting of ³²P, ⁴⁷Sc, ⁶⁷Cu, ⁹⁰Y, ¹⁰⁵Rh, ¹²⁵I, ¹³¹I, ^{117m}Sn, ¹⁵³Sm, ¹⁶⁶Dy, ¹⁷⁵Yb, ¹⁸⁶Re, ¹⁸⁸Re, ¹⁹⁴Os, ²¹¹At, ²¹²Bi, ²¹³Bi, ²²⁵Ac, and mixtures thereof.

28. A method for treating a mammal having an AF-20-expressing cancer comprising administering to the mammal a therapeutically effective amount of a composition according to claim 24.

29. The method according to claim 28, wherein the composition is administered post-operatively.

30. A composition for detecting cancer comprising a diagnostically effective amount of the humanized antibody or humanized antibody fragment of claim 2.

31. The composition of claim 30, wherein the humanized antibody or humanized antibody fragment is, directly or indirectly, associated with or linked to a detectable label.

32. The composition of claim 31, wherein the detectable label is selected from the group consisting of a radionuclide, fluorescer, enzyme, enzyme substrate, enzyme cofactor, enzyme inhibitor, ligand, and mixtures thereof.

33. The composition of claim 32, wherein the radionuclide is selected from the group consisting of ³H, ¹¹C, ¹⁴C, ¹⁸F, ⁶⁴Cu, ⁷⁶Br, ⁸⁶Y, ^{99m}Tc, ¹¹¹In, ¹²³I, ¹⁷⁷Lu, and mixtures thereof.

34. A method for immunodetection of AF-20-expressing cancer cells comprising contacting the cancer cells with the composition of claim 30.

35. The method of claim 34, wherein the humanized antibody or humanized antibody fragment of the composition is bound to a solid support.

36. A method of immunodetection of AF-20-expressing cancer cells in a mammal comprising administering to the mammal a diagnostically effective amount of the composition according of claim 30.

37. The method of claim 36, wherein said immunodetection is in vivo tumor imaging.

38. A method of treating cancer comprising: (i) intravenously administering a radionuclide-labeled humanized antibody, or humanized antibody fragment of claim 6; (ii) detecting tumor cells using a radionuclide activity probe; and (iii) removing the detected tumor cells by surgical excision.

39. The method of claim 38, wherein the radionuclide is selected from the group consisting of ³H, ¹¹C, ¹⁴C, ¹⁸F, ⁶⁴Cu, ⁷⁶Br, ⁸⁶Y, ^{99m}Tc, ¹¹¹In, ¹²³I, ¹⁷⁷Lu, and mixtures thereof.


SEQUENCE LISTING (49 sequences)

SEQ ID NO: 1 - DNA, 822 bp, Mus sp.
  CDS (122)..(160)
  CDS (249)..(620)
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaggtccaactgcagcagtctggacctgacctggtgaagcctggggcttcagtgaggatatcctgcaaggcttctggctacaccttcgcaggccactatgtacactgggtgaagcagaggcctggacggggacttgagtggattggatggattttccctggaaaggtaaatactaagtacaatgagaagttcaagggcaaggccacattgactgcagacaaatcctccagcacagcctacatgcagctcagcagcctgacctctgaggactctgcggtctatttctgtgcaagagttggatatgattacccgtactactttgactactggggccaaggcaccactctcacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcc

SEQ ID NO: 2 - protein, 13 aa, Mus sp.
MGWSCIILFLVAT

SEQ ID NO: 3 - protein, 124 aa, Mus sp.
GVHSQVQLQQSGPDLVKPGASVRISCKASGYTFAGHYVHWVKQRPGRGLEWIGWIFPGKVNTKYNEKFKGKATLTADKSSSTAYMQLSSLTSEDSAVYFCARVGYDYPYYFDYWGQGTTLTVSS

SEQ ID NO: 4 - DNA, 620 bp, Mus sp.
  CDS (122)..(160)
  CDS (249)..(581)
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactccgacatcttgctgactcagtctccagccatcctgtctgtgagtccaggagacagagtcagtttctcctgcagggccagtcagagcattggcacaagcatacactggtatcagcaaagaacaaatggttctccaaggcttctcataaagtatgcttctgagtctatctctgggattccttccaggtttagtggcagtggatcagggacagattttactcttagcattaacagtgtggagtctgaagatgttgcagattattactgtcaacaaagtagtagctggccattcacgttcggctcggggacaaagttggaaataaaacgtgagtagaatttaaactttgcttcctcagttggatcc

SEQ ID NO: 5 - protein, 13 aa, Mus sp.
MGWSCIILFLVAT

SEQ ID NO: 6 - protein, 111 aa, Mus sp.
GVHSDILLTQSPAILSVSPGDRVSFSCRASQSIGTSIHWYQQRTNGSPRLLIKYASESISGIPSRFSGSGSGTDFTLSINSVESEDVADYYCQQSSSWPFTFGSGTKLEIK

SEQ ID NO: 7 - protein, 13 aa, Mus sp.
DLVKPGASVRISC

SEQ ID NO: 8 - protein, 13 aa, Mus sp.
ASVRISCKASGYT

SEQ ID NO: 9 - protein, 13 aa, Mus sp.
HYVHWVKQRPGQG

SEQ ID NO: 10 - protein, 13 aa, Mus sp.
HWVKQRPGQGLEW

SEQ ID NO: 11 - protein, 13 aa, Mus sp.
TKYNEKFKGKATL

SEQ ID NO: 12 - protein, 13 aa, Mus sp.
TAYMQLSSLTSED

SEQ ID NO: 13 - protein, 13 aa, Mus sp.
MQLSSLTSEDSAV

SEQ ID NO: 14 - protein, 13 aa, Mus sp.
SSLTSEDSAVYFC

SEQ ID NO: 15 - protein, 13 aa, Mus sp.
DYWGQGTTLTVSS

SEQ ID NO: 16 - protein, 13 aa, Mus sp.
DILLTQSPAILSV

SEQ ID NO: 17 - protein, 13 aa, Mus sp.
ILLTQSPAILSVS

SEQ ID NO: 18 - protein, 13 aa, Mus sp.
LSVSPGDRVSFSC

SEQ ID NO: 19 - protein, 13 aa, Mus sp.
DRVSFSCRASQSI

SEQ ID NO: 20 - protein, 13 aa, Mus sp.
HWYQQRTNGSPRL

SEQ ID NO: 21 - protein, 13 aa, Mus sp.
FTLSINSVESEDV

SEQ ID NO: 22 - protein, 13 aa, Mus sp.
NSVESEDVADYYC

SEQ ID NO: 23 - protein, 13 aa, Mus sp.
EDVADYYCQQSSS

SEQ ID NO: 24 - protein, 120 aa, Mus sp.
QVQLQQSGPDLVKPGASVRISCKASGYTFAGHYVHWVKQRPGQGLEWIGWILPGKVNTKYNEKFKGKATLTADKSSSTAYMQLSSLTSEDSAVYFCARVGYDYPYYFDYWGQGTTLTVSS

SEQ ID NO: 25 - protein, 120 aa, Artificial Sequence
  Description of Artificial Sequence Synthetic VH variant sequence
QVQLQQSGPDLAKPGASARISCKASGYTFAGHYVHWTKQRPGQGLEWIGWILPGKTNTKYNEKFKGKTTLTADKSSSTAYMQLSSETSEDSAVYFCARVGYDYPYYFDYWGQGTTVTVSS

SEQ ID NO: 26 - protein, 120 aa, Artificial Sequence
  Description of Artificial Sequence Synthetic VH variant sequence
QVQLQQSGPDLAKPGASARISCKASGYTFAGHYVHWTKQRPGQGLEWIGWILPGKTNTKYNEKFKGKTTLTADKSSSTAYMELSSLRSEDTAVYYCARVGYDYPYYFDYWGQGTTVTVSS

SEQ ID NO: 27 - protein, 120 aa, Artificial Sequence
  Description of Artificial Sequence Synthetic VH variant sequence
QVQLQQSGPDLAKPGASARISCKASGYTFAGHYVHWTKQRPGQGLEWIGWILPGKVNTKYNEKFKGKTTLTADKSSSTAYMELSSLRSEDTAVYYCARVGYDYPYYFDYWGQGTTVTVSS

SEQ ID NO: 28 - protein, 120 aa, Artificial Sequence
  Description of Artificial Sequence Synthetic VH variant sequence
QVQLQQSGPDLAKPGASARISCKASGYTFAGHYVHWVKQRPGQGLEWIGWILPGKVNTKYNEKFKGKTTLTADKSSSTAYMELSSLRSEDTAVYYCARVGYDYPYYFDYWGQGTTVTVSS

SEQ ID NO: 29 - protein, 120 aa, Artificial Sequence
  Description of Artificial Sequence Synthetic VH variant sequence
QVQLQQSGPDLAKPGASARISCKASGYTFAGHYVHWVKQRPGQGLEWIGWILPGKVNTKYNEKFKGKTTLTADKSSSTAYMELSSLRSEDTAVYFCARVGYDYPYYFDYWGQGTTVTVSS

SEQ ID NO: 30 - protein, 120 aa, Artificial Sequence
  Description of Artificial Sequence Synthetic VH variant sequence
QVQLQQSGPDLAKPGASARISCKASGYTFAGHYVHWVKQRPGQGLEWIGWILPGKVNTKYNEKFKGKATLTADKSSSTAYMELSSLRSEDTAVYFCARVGYDYPYYFDYWGQGTTVTVSS

SEQ ID NO: 31 - protein, 120 aa, Artificial Sequence
  Description of Artificial Sequence Synthetic VH variant sequence
QVQLQQSGPDLAKPGASARISCKASGYTFAGHYVHWVKQRPGQGLEWIGWILPGKVNTKYNEKFKGKATLTADKSSSTAYMELSSLTSEDTAVYFCARVGYDYPYYFDYWGQGTTVTVSS

SEQ ID NO: 32 - protein, 107 aa, Mus sp.
DILLTQSPAILSVSPGDRVSFSCRASQSIGTSIHWYQQRTNGSPRLLIKYASESISGIPSRFSGSGSGTDFTLSINSVESEDVADYYCQQSSSWPFTFGSGTKLEIK

SEQ ID NO: 33 - protein, 107 aa, Artificial Sequence
  Description of Artificial Sequence Synthetic VK variant sequence
DIVMTQSPAIVSASPGDRASFSCRASQSIGTSAHWYQQRTNSSPRLLIKYASEVISGIPSRFSGSGSGTDFTMSINSTESEDTADYYCQQSSSWPFTFGSGTKLEIK

SEQ ID NO: 34 - protein, 107 aa, Artificial Sequence
  Description of Artificial Sequence Synthetic VK variant sequence
DIVMTQSPAIVSASPGDRASFSCRASQSIGTSIHWYQQRTNSSPRLLIKYASEVISGIPSRFSGSGSGTDFTMSINSTESEDTADYYCQQSSSWPFTFGSGTKLEIK

SEQ ID NO: 35 - protein, 107 aa, Artificial Sequence
  Description of Artificial Sequence Synthetic VK variant sequence
DIVMTQSPAIVSASPGDRASFSCRASQSIGTSIHWYQQRTNSSPRLLIKYASESISGIPSRFSGSGSGTDFTMSINSTESEDTADYYCQQSSSWPFTFGSGTKLEIK

SEQ ID NO: 36 - protein, 107 aa, Artificial Sequence
  Description of Artificial Sequence Synthetic VK variant sequence
DIVMTQSPAIVSASPGDRASFSCRASQSIGTSIHWYQQRTNSSPRLLIKYASESISGIPSRFSGSGSGTDFTLSINSTESEDTADYYCQQSSSWPFTFGSGTKLEIK

SEQ ID NO: 37 - protein, 107 aa, Artificial Sequence
  Description of Artificial Sequence Synthetic VK variant sequence
DIVMTQSPAIVSASPGDRASFSCRASQSIGTSIHWYQQRTNGSPRLLIKYASESISGIPSRFSGSGSGTDFTLSINSTESEDTADYYCQQSSSWPFTFGSGTKLEIK

SEQ ID NO: 38 - DNA, 822 bp, Artificial Sequence
  Description of Artificial Sequence Synthetic VH variant sequence
  CDS (122)..(160)
  CDS (249)..(620)
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaggtccaactgcagcagtctggacctgacctggcgaagcctggggcttcagcgaggatatcctgcaaggcttctggctacaccttcgcaggccactatgtacactggactaagcagaggcctggacggggacttgagtggattggatggattttccctggaaagacaaatactaagtacaatgagaagttcaagggcaagaccacattgactgcagacaaatcctccagcacagcctacatggagctcagcagcctgagatctgaggacactgcggtctattattgtgcaagagttggatatgattacccgtactactttgactactggggccaaggcaccactgtcacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcc

SEQ ID NO: 39 - protein, 13 aa, Artificial Sequence
  Description of Artificial Sequence Synthetic VH variant sequence
MGWSCIILFLVAT

SEQ ID NO: 40 - protein, 124 aa, Artificial Sequence
  Description of Artificial Sequence Synthetic VH variant sequence
GVHSQVQLQQSGPDLAKPGASARISCKASGYTFAGHYVHWTKQRPGRGLEWIGWIFPGKTNTKYNEKFKGKTTLTADKSSSTAYMELSSLRSEDTAVYYCARVGYDYPYYFDYWGQGTTVTVSS

SEQ ID NO: 41 - DNA, 620 bp, Artificial Sequence
  Description of Artificial Sequence Synthetic VK variant sequence
  CDS (122)..(160)
  CDS (249)..(581)
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactccgacatcgtgatgactcagtctccagccatcgtgtctgcgagtccaggagacagagccagtttctcctgcagggccagtcagagcattggcacaagcgcacactggtatcagcaaagaacaaatagttctccaaggcttctcataaagtatcgttctgaggttatctctgggattccttccaggtttagtggcagtggatcagggacagattttactatgagcattaacagtacggagtctgaagatactgcagattattactgtcaacaaagtagtagctggccattcacgttcggctcggggacaaagttggaaataaaacgtgagtagaatttaaactttgcttcctcagttggatcc

SEQ ID NO: 42 - protein, 13 aa, Artificial Sequence
  Description of Artificial Sequence Synthetic VK variant sequence
MGWSCIILFLVAT

SEQ ID NO: 43 - protein, 111 aa, Artificial Sequence
  Description of Artificial Sequence Synthetic VK variant sequence
GVHSDIVMTQSPAIVSASPGDRASFSCRASQSIGTSAHWYQQRTNSSPRLLIKYRSEVISGIPSRFSGSGSGTDFTMSINSTESEDTADYYCQQSSSWPFTFGSGTKLEIK

SEQ ID NO: 44 - protein, 11 aa, Artificial Sequence
  Description of Artificial Sequence Synthetic amino acid sequence
RASQSIGTSIH

SEQ ID NO: 45 - protein, 7 aa, Artificial Sequence
  Description of Artificial Sequence Synthetic amino acid sequence
YASESIS

SEQ ID NO: 46 - protein, 9 aa, Artificial Sequence
  Description of Artificial Sequence Synthetic amino acid sequence
QQSSSWPFT

SEQ ID NO: 47 - protein, 10 aa, Artificial Sequence
  Description of Artificial Sequence Synthetic amino acid sequence
GYTFAGHYVH

SEQ ID NO: 48 - protein, 17 aa, Artificial Sequence
  Description of Artificial Sequence Synthetic amino acid sequence
WIFPGKVNTKYNEKFKG

SEQ ID NO: 49 - protein, 11 aa, Artificial Sequence
  Description of Artificial Sequence Synthetic amino acid sequence
VGYDYFYYFDY